(19) United States
(12) Patent Application Publication
(10) Pub. No.: US 2024/0216383 A1
(43) Pub. Date: Jul. 4, 2024

(54) FORMULATIONS AND METHODS FOR TREATING ERECTILE DYSFUNCTION

(71) Applicant: Strategic Drug Solutions, Inc., Chino Hills, CA (US)

(72) Inventors: Moses Chow, Chino Hills, CA (US); Sheryl L. Chow, Chino Hills, CA (US)

(21) Appl. No.: 17/999,826

(22) PCT Filed: Dec. 1, 2021
(86) PCT No.: PCT/US2021/061488

§ 371 Date: Dec. 1, 2022

Related U.S. Application Data
(63) Continuation-in-part of application No. PCT/US21/34334, filed on May 26, 2021.
(60) Provisional application No. 63/029,881, filed on May 26, 2020.

(51) Int. Cl. A61K 31/53; A61K 9/00; A61K 31/4985; A61K 31/519; A61K 47/10

(52) U.S. Cl. CPC A61K 31/53; A61K 9/0043; A61K 9/006; A61K 31/4985; A61K 31/519; A61K 47/10

(57) ABSTRACT

The formulation disclosed herein has beneficial properties in enhancing the solubility and permeability (flux) of at least one of vardenafil, sildenafil, and/or related phosphodiesterase inhibitors or other ionizable compounds. In particular, the formulation confers beneficial properties for compounds in crossing the mucosal membrane, leading to an effective plasma drug concentration. In some embodiments, the formulation comprises an organic-aqueous solvent, which may include at least one of an alcohol, a polyether, diethylene glycol monoethyl ether, a medium chain glyceride, one or more saturated polyglycolyzed C8-C10 glyceride, and/or organic salts, or any combination thereof. In some embodiments, the formulation has a pH of about 3.5 to about 8.0. Also disclosed herein is a use for the formulation in administering one or more compound for the treatment of erectile dysfunction or other diseases, wherein the one or more compound is administered transmucosally (intranasally or sublingually).

### RELATED APPLICATION

This application is a continuation-in-part of International Application No. PCT/US21/34334, filed May 26, 2021.

### FIELD OF THE INVENTION

There exists a need for improved formulations and methods for treating erectile dysfunction. The present technology generally relates to formulations and methods of treating erectile dysfunction with phosphodiesterase inhibitors, but can be applied to other drugs in treating different disease conditions using transmucosal administration, for example sublingual or intranasal administrations.

### BACKGROUND OF THE INVENTION

Erectile dysfunction is considered the most common form of sexual dysfunction in men, and becomes increasingly common with age. It's estimated that approximately 50% of men between the ages of 40-70, and 70% of men over the age of 70, deal with erectile dysfunction. Because erectile dysfunction can be caused by one or more of neurological, vascular, endocrinological, or psychological factors, the condition is not limited to elderly men. Other risk factors such as cardiovascular disease, hypertension, diabetes, hypercholesterolemia, and smoking have been strongly associated with an increased prevalence of erectile dysfunction. Consequently, there is an increasing need for the effective treatment of erectile dysfunction.

### SUMMARY OF THE INVENTION

There exists a need for compositions and methods to sufficiently solubilize and allow for sufficient permeation of phosphodiesterase inhibitors, including, for example, vardenafil, sildenafil, and tadalafil. Disclosed herein, in some embodiments, are organic-aqueous mixtures that are relatively safe or well-tolerated by human subjects as well as capable of sufficiently solubilizing a phosphodiesterase inhibitor. In some embodiments, organic aqueous mixtures are screened and identified based on solubility of the phosphodiesterase inhibitor. In some embodiments, the phosphodiesterase inhibitor is vardenafil. In some embodiments, the phosphodiesterase inhibitor is sildenafil. In some embodiments, the phosphodiesterase inhibitor is tadalafil. In some embodiments, the pH and the permeation effect are determined.

Described herein, in some embodiments, are methods to identify formulations for enhancing solubility and permeation of one or more phosphodiesterase inhibitor across a mucosal membrane, comprising: (a) one or more phosphodiesterase inhibitor; and (b) an organic-aqueous solvent comprising an alcohol, a glycol, diethylene glycol monoethyl ether, a medium chain glyceride, one or more saturated polyglycolyzed C8-C10 glyceride, or a combination thereof; wherein the formulation has a pH of about 3.5 to about 8.0 and wherein the organic-aqueous solvent enhances solubility of the one or more phosphodiesterase inhibitor relative to solubility of the one or more phosphodiesterase inhibitor in water. In some embodiments, the organic-aqueous solvent comprises an alcohol. In some embodiments, the formulations described herein comprise one or more weak salts. Exemplary weak salts include, for example, citric acid, tartaric acid, acetic acid, furmaric acid, lactic acid, ammonium chloride or similar organic salts, and others. In some embodiments, the formulations described herein comprise N-methyl pryrrolidone (NMP), Tween 80 or similar organic compounds. In some embodiments, the formulations described herein comprise a weak salt such as citric acid, tartaric acid, acetic acid, furmaric acid, lactic acid, ammonium chloride or similar organic salts, and others, or N-methyl pryrrolidone (NMP), Tween 80 or similar organic compounds in combination with one or more alcohol, a polyether, diethylene glycol monoethyl ether, a medium chain glyceride, one or more saturated polyglycolyzed C8-C10 glyceride, or a combination thereof. In some embodiments, the alcohol is ethanol or glycerol. In some embodiments, the ethanol is present at a concentration of 5% to 40%. In some embodiments, the ethanol is present at a concentration of 12%, 25%, or 30%. In some embodiments, the organic-aqueous solvent comprises a polyether. In some embodiments, the polyether is polyethylene glycol. In some embodiments, the polyethylene glycol is PEG 6000 or PEG 400. In some embodiments, the polyethylene glycol is present at a concentration of 1% to 20%. In some embodiments, the polyethylene glycol is present at a concentration of 5%. In some embodiments, the formulation has a pH of about 3.5 to about 8.0. In some embodiments, the phosphodiesterase inhibitor is vardenafil, sildenafil, tadalafil, or a combination thereof. In some embodiments, the phosphodiesterase inhibitor is vardenafil. In some embodiments, the phosphodiesterase inhibitor is sildenafil. In some embodiments, the phosphodiesterase inhibitor is tadalafil.

Described herein, in some embodiments, are methods of treating erectile dysfunction of a subject in need thereof, comprising contacting a mucosal membrane of the subject with a formulation disclosed herein, thereby treating the erectile dysfunction of the subject. In some embodiments, contacting the mucosal membrane comprises intranasal administration. In some embodiments, contacting the mucosal membrane comprises sublingual administration.

Described herein, in some embodiments, are methods of preparing a formulation for treating erectile dysfunction of a subject, comprising: (a) adding one or more phosphodiesterase inhibitor to an organic-aqueous solvent comprising an alcohol, a polyether, diethylene glycol monoethyl ether, a medium chain glyceride, one or more saturated polyglycolyzed C8-C10 glyceride, or a combination thereof; (b) adjusting the pH of the organic-aqueous solvent comprising the one or more phosphodiesterase inhibitor to about 3.5 to about 8.0 wherein treating the erectile dysfunction comprises contacting a mucosal membrane of the subject with the formulation. In some embodiments, the formulations described herein comprise one or more weak salts. Exemplary weak salts include, for example, citric acid, tartaric acid, acetic acid, furmaric acid, lactic acid, ammonium chloride or similar organic salts, and others. In some embodiments, the formulations described herein comprise N-methyl pryrrolidone (NMP), Tween 80 or similar organic compounds. In some embodiments, the formulations described herein comprise a weak salt such as citric acid, tartaric acid, acetic acid, furmaric acid, lactic acid, ammonium chloride or similar organic salts, and others, or N-methyl pryrrolidone (NMP), Tween 80 or similar organic compounds in combination with one or more alcohol, a polyether, diethylene glycol monoethyl ether, a medium chain glyceride, one or more saturated polyglycolyzed C8-C10 glyceride, or a combination thereof. In some embodiments, solubility of the one or more phosphodiesterase inhibitor is increased in the organic-aqueous solvent relative to solubility of the one or more phosphodiesterase inhibitor in water. In some embodiments, permeation of the one or more phosphodiesterase inhibitor across the mucosal membrane is increased in the organic-aqueous solvent relative to permeation of the one or more phosphodiesterase inhibitor in water. In some embodiments, permeation of the one or more phosphodiesterase inhibitor across an artificial membrane in vitro is increased in the organic-aqueous solvent relative to permeation of the one or more phosphodiesterase inhibitor in water. In some embodiments, bioavailability of the one or more phosphodiesterase inhibitor is increased in the organic-aqueous solvent relative to bioavailability of the one or more phosphodiesterase inhibitor in water. In some embodiments, the organic-aqueous solvent comprises an alcohol. In some embodiments, the alcohol is ethanol or glycerol. In some embodiments, the ethanol is present at a concentration of 5% to 40%. In some embodiments, the ethanol is present at a concentration of 12%, 25%, or 30%. In some embodiments, the organic-aqueous solvent comprises a polyether. In some embodiments, the polyether is polyethylene glycol. In some embodiments, the polyethylene glycol is PEG 6000 or PEG 400. In some embodiments, the polyethylene glycol is present at a concentration of 1% to 20%. In some embodiments, the polyethylene glycol is present at a concentration of 5%. In some embodiments, the formulation has a pH of about 3.5 to about 8.0. In some embodiments, the phosphodiesterase inhibitor is vardenafil, sildenafil, tadalafil, or a combination thereof. In some embodiments, the phosphodiesterase inhibitor is vardenafil. In some embodiments, the phosphodiesterase inhibitor is sildenafil. In some embodiments, the phosphodiesterase inhibitor is tadalafil.

### BRIEF DESCRIPTION OF THE DRAWINGS

FIG. 1 illustrates a calibration curve of vardenafil concentration in purified water (y=0.00853x+0.006553, R²=0.9962).

FIG. 2 illustrates stable soluble concentrations of vardenafil HCl trihydrate in water (mg/ml) at different pH values using an HPLC method.

FIG. 3 illustrates simultaneous determination of solubility of saturated solutions of vardenafil HCl trihydrate (mg/ml) in water, 12% alcohol and 30% alcohol.

FIG. 4 illustrates a relationship between the apparent permeability coefficient (Papp) for vardenafil at 6 and 12 hours, as per PAMPA study.

FIG. 5 illustrates comparisons of the effect of pH on the Papp of various formulations (panel a), the effect of pH on the Jss of various formulations (panel b)

FIG. 6 illustrates comparisons of vardenafil Papp values in different solutions using either PAMPA or the Calu-3 cell line model. Open circle represents water solution. Closed circles represent EtOH(12%), PEG400(15%), NMP(10%), Calcium Lactate (5%) EtOH/PEG400(12%/15%), EtOH/Calcium Lactate( ) 2%/5%). Only the lowest Papp (lower left), representing PEG400(15%), is significantly lower than that in water (p<0.05 t-test)

FIG. 7 illustrates a representative curve of vardenafil concentration in blood plasma of subject 11 (“S11”) following administration through either intranasal (IN) or oral (PO).

FIG. 8 illustrates the effect of pH on sildenafil flux (Jss) based on PAMPA study

FIG. 9 illustrates the comparison of Papp values in different solutions using either PAMPA or the Calu-3 cell line model. Open circle represents water solution. Closed circles represent acetic acid/NMP/calcium lactate(5/10/3.5%), acetic acid/calcium lactate (5/3.5%), acetic acid/calcium lactate (1/3.5%), NMP (10%), calcium lactate (3.5%).

FIG. 10 illustrates comparisons of vardenafil HCl trihydrate permeation over 24 hours in water (columns 1-5), 12% ethanol-aqueous solution (columns 6-10), and 30% ethanol-aqueous solution (columns 11-15). Saturated concentrations were used.

FIG. 11 illustrates comparisons of vardenafil permeation using saturated concentrations in glycerin (glycerol), polyethylene glycol (PEG), and PEG-ethanol (EtHO) mixtures.

FIG. 12 illustrates a calibration curve of vardenafil concentration with purified water (y=0481x+0.0033; R²=0.9994).

FIG. 13 illustrates a calibration curve of vardenafil in 25% ethanol-aqueous mixture (y=0.583x+0.0043, R²=0.9945).

FIG. 14 illustrates simultaneous determination of the saturated solubility of vardenafil API in water, 12% and 30% alcohol (EtOH).

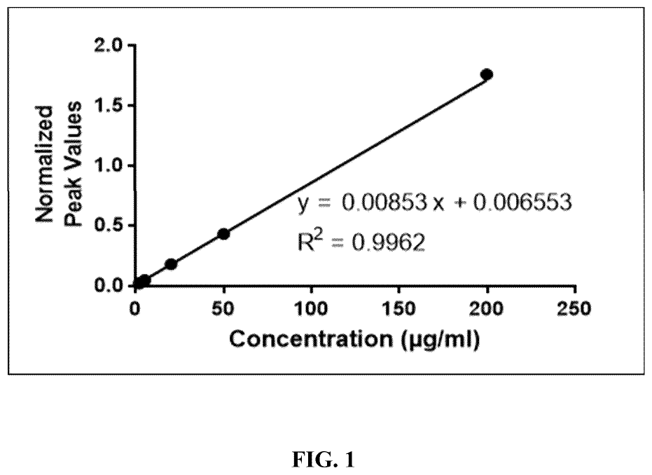

### DETAILED DESCRIPTION OF VARIOUS EMBODIMENTS

The present invention relates to formulations and methods of optimizing solubility and permeation of phosphodiesterase inhibitors across a mucosal membrane. The formulations and methods provided herein can be used for the treatment of erectile dysfunction, for example. It will be understood to one skilled in the art that the formulation and methodology disclosed herein can be applicable to any ionizable compound, including acidic and basic compounds, and drugs or other compounds that are not phosphodiesterase inhibitors. Non-limiting examples of ionizable compounds that may be administered across a mucosal membrane through the formulations disclosed herein include levodopa, chlorothiazide, furosemide, ibuprofen, levodopa, warfarin, acetazolamide, phenytoin, theophylline, chloropropamide, bumetanide, diazepam, allopurinol, alprenolol, amphetamine, atropine, codeine, codeine, lidocaine, metoprolol, epinephrine, imipramine, methadone, methamphetamine, morphine, nicotine, norepinephrine, and pilocarpine.

Normal penile erection results from the influx of blood and relaxation of smooth muscle in the penis. The process is mediated by a spinal reflex, the L-arginine-nitric oxide-guanylyl cyclase-cyclic guanosine monophosphate (cGMP) pathway, and sensory and mental stimuli. Nerves and endothelial cells directly release nitric oxide in the penis, where it stimulates guanylyl cyclase to produce cGMP and lowers intracellular calcium levels. This triggers relaxation of arterial and trabecular smooth muscle, leading to arterial dilatation, venous constriction, and erection. The balance between factors that stimulate contraction and relaxation determines the tone of penile vasculature and the smooth muscle of the corpus cavernosum.

Phosphodiesterase 5 (PDE5) is the predominant phosphodiesterase in the corpus cavernosum. The catalytic site of PDE5 normally degrades cGMP, and PDE5 inhibitors such as sildenafil potentiate endogenous increases in cGMP by inhibiting its breakdown at the catalytic site. Phosphorylation of PDE5 increases its enzymatic activity as well as the affinity of its allosteric (noncatalytic/GAF domains) sites for cGMP. Binding of cGMP to the allosteric site further stimulates enzymatic activity. Thus phosphorylation of PDE5 and binding of cGMP to the noncatalytic sites mediate negative feedback regulation of the cGMP pathway.

Sildenafil, tadalafil, and vardenafil are approved by the FDA for the treatment of erectile dysfunction. These drugs all act as phosphodiesterase inhibitors. A phosphodiesterase inhibitor is a drug that blocks one or more of five subtypes of the enzyme phosphodiesterase (PDE), thereby preventing the inactivation of the intracellular second messengers cyclic adenosine monophosphate (cAMP) and cyclic guanosine monophosphate (cGMP). Given that phosphodiesterases are responsible for degradation of cyclic guanosine monophosphate (cGMP) which triggers smooth muscle relaxation and erection during sexual stimulation, inhibition of one or more phosphodiesterase by these drugs will enhance erectile function by increasing cGMP.

### Sildenafil (Viagra)

The effective dose of sildenafil (Viagra) is 25-100 mg once a day as needed. The active ingredient is sildenafil citrate. Its mean maximum plasma concentration is about 60 min (range 30-90 min) and its absolute bioavailability is about 41%. The drug is mostly metabolized by cytochrome P450 3A4 (CYP3A4), with a half-life of about 4 h (1).

### Tadalafil (Cialis)

The effective dose of tadalafil (Cialis) is a 5-20 mg once a day as needed. Its active ingredient is tardafil. The mean time (Tmax) for maximum plasma concentration is about 2 h (range 30 min-6 h) following a single dose (2). The drug is mostly metabolized by CYP3A4 to a catechol metabolite which is further glucuronidated. The mean terminal half-life is about 17.5 h in healthy subjects (2). The absolute bioavailability after oral administration has not been reported to exceed 80% (3).

### Vardenafil (Levitra)

The standard recommended dose of vardenafil (Levitra) is a 10-20 mg tablet once a day as needed. Its active ingredient is vardenafil hydrochloride trihydrate. The mean time (Tmax) for maximum plasma concentration is about 60 min (30 min-2 h) and its absolute bioavailability after oral administration is about 15%. The drug is mostly metabolized by CYP3A4 and the MI metabolite accounts for about 7% of total pharmacologic activity. The terminal half-life of vardenafil or the MI metabolite is about 4-5 h, and the onset of the therapeutic effect is about 30 min (4).

Each of these three phosphodiesterase inhibitor drugs is approved by the FDA for erectile dysfunction and has a mean time (Tmax) for maximum concentration at about 60 minutes or longer, with an early Tmax at 30 min. Thus, the onset of action for these drugs is usually 30 min or later, with maximum effect at 1 h. Since their aqueous solubility at pH 4.0-7 (close to physiologic pH range at nasal and sublingual membranes) (5-7) is low, these drugs are not suitable for administration as an aqueous solution when administered sublingually or intranasally to achieve a rapid effect.

To achieve good permeation and/or absorption at sublingual or nasal sites, a drug must have a small molecular weight (<1 kD), a good membrane partition coefficient (with a good log P), and good aqueous solubility (7-9). When administered intranasally or sublingually, the thin nasal and sublingual membranes can provide more rapid absorption than absorption upon oral administration (6, 7). In addition, intranasal and sublingual routes of administration can bypass liver first metabolism and can yield greater bioavailability than bioavailability upon oral administration (10-11). However, the aqueous solubility of the three phosphodiesterase inhibitor drugs is low at pH 4.0-7.0, which is a major obstacle for efficient permeation and/or absorption at nasal or sublingual sites. To optimize mucosal permeation and/or absorption via the sublingual or intranasal routes of administration, a suitable solvent (such as an organic-aqueous mixture) that can improve solubility and achieving similar or better permeability as that at suitable pH at these sites. However there is no reliable method to predict the solubility and permeability in these solvents at optimal pH.

### Definitions

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as is commonly understood by one of skill in the art to which this invention belongs. “About” as used herein when referring to a measurable value such as an amount, a temporal duration, and the like, is meant to encompass variations of ±20% or ±10%, or ±5%, or even ±1% from the specified value, as such variations are appropriate for the disclosed compositions or to perform the disclosed methods.

Definition of standard chemistry terms may be found in reference works, including Carey and Sundberg “ADVANCED ORGANIC CHEMISTRY 4TH ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Reactions and purification techniques can be performed e.g., using kits of manufacturer's specifications or as commonly accomplished in the art or as described herein. The foregoing techniques and procedures can be generally performed of methods known in the art and as described in various general and more specific references that are cited and discussed throughout the present specification.

As used herein, the terms “permeation” or “absorption,” unless specified otherwise, mean “penetration” of the active compound of a medicament through a mucosa. The terms “permeation” and “absorption” may be used interchangeably.

As used herein, the terms “transmucosal” or “across a mucosal membrane,” unless specified otherwise, mean any route of administration via a mucosal membrane. Examples include, but are not limited to, sublingual, nasal, vaginal and rectal administration of a medicament or an active compound of a medicament.

As used herein, the term “phosphodiesterase inhibitor” refers to any drug that blocks one or more subtype of the enzyme phosphodiesterase (PDE), thereby preventing the inactivation of the intracellular second messengers cyclic adenosine monophosphate (cAMP) and cyclic guanosine monophosphate (cGMP) by the respective PDE subtype(s). The term “phosphodiesterase inhibitor” can refer to an inhibitor of PDE1, PDE2, PDE3, PDE4, PDE5, PDE6, PDE7, PDE8, PDE9, PDE10, PDE11 and/or PDE12. Phosphodiesterase inhibitors include selective and non-selective inhibitors.

As used herein, the term “subject” means animal and human.

The term “environment” or “environment of an administration” means an environment where an active compound of a medicament is absorbed by permeation across the mucosa. For example, when the administration is performed sublingually, the environment is saliva, which contains the drug and is “bathing” the sublingual mucosal membrane.

The method of embodiments which provides an environment with a certain pH includes providing the environment with a preferable pH during the administration of the medicament, and making a suitable formulation of the medicament in such a way that the medicament itself can provide the environment with a desired pH. In some embodiments, the latter is preferred. In this case, buffering agents are preferably involved in the formulation.

The embodiment described herein can include calculating an estimated range of vardenafil quantity (from minimum quantity of vardenafil API to 2-fold representing minimum effective dose to 2 fold the minimum dose) that needs to be solubilized and then permeated or absorbed across mucosal membrane to achieve a therapeutic effective concentration.

The embodiments described herein can include various formulations or compositions dependent on the dosage forms or routes of administration. For example, if a formulation or composition comprising a medicament is administered sublingually, it can be in the form of tablets, pills, pellets, powders, liquid or sprays. Examples of other suitable formulations or compositions include, but are not limited to, ointments, capsules, solutions, syrups, drops, granules and suppositories. In any formulation or composition, the medicament can include a therapeutically effective amount of an active compound or a pharmaceutically acceptable form thereof or either entity and a pharmaceutically acceptable carrier. As another example, if a formulation or composition comprising a medicament is administered intranasally, the formulation or composition can be in liquid form. Suitable liquid forms for intranasal administration are nasal sprays and nasal drops, for example.

In some embodiments, the one or more phosphodiesterase inhibitor is administered sublingually. For sublingual administration of the one or more phosphdiesterase inhibitor, a formulation or composition can be in any of the forms described above. Any method of making tablets, pills, pellets, powders, liquid or sprays for sublingual administration can be used. To make tablets, granulated powder is pressed into a small tablet, for example. The tablet can disintegrate when mixed with saliva, resulting in solubilization and absorption of the drug. To obtain a desired pH range for permeation and/or absorption of the drug, a tablet formulation is made taking into account mixing with saliva, for example.

Alcohol powder can be used to make tablets for sublingual administration. As another example, polyethylene glycol (PEG) can be used to make tablets for sublingual administration. Both alcohol powder and PEG are miscible with water. Exemplary liquid PEGs that can be used include, but are not limited to, PEG200, PEG400, and PEG600. Exemplary waxy or solid PEGs that can be used include, but are not limited to PEGs with an average molecular weight of greater than about 600 g/mol (PEG600), such as PEG3000, PEG3350, PEG4000, PEG6000, and PEG8000.

In some embodiments, the one or more phosphodiesterase inhibitor is administered intranasally. For intranasal administration of the one or more phosphdiesterase inhibitor, a formulation or composition can be in any of the forms described above, including a nasal spray or liquid drops, for example. A special device can be used for intranasal or sublingual administration of a set volume. Exemplary volumes for such devices can be in the range of 10 μl to 1.6 ml, which can be delivered to each of two nostrils. Further exemplary volumes can be in the range of 25 μl to 1.0 ml, 50 μl to 800 μl, 75 μl to 600 μl, 100 μl to 500 μl or 200 μl to 300 μl, per nostril for at least one nostril. Devices for intranasal administration are commercially available from Aptar, for example.

Intranasal (IN) drug administration, e.g. via nasal spray, is a convenient route of administration. This route of administration can achieve the following advantages relative to oral drug administration: (a) produce faster effect, and (b) smaller amount of drug exposure to achieve equal effect, and (c) administering without the need of water for swallowing. These advantages of IN administration is possible because of the leaky epithelium lining the nasal mucosa (as compared to intestinal epithelium), extensive vascular supply, relatively large surface area (about 9.6 m² including microvilli) and avoidance of first pass metabolism (3-9). The relatively large surface area for drug absorption via IN route is also an advantage over sublingual route. Although the sublingual route can also provide a rapid onset of effect, its much smaller surface area (26 cm²) is a limitation for drug absorption that would lead to inadequate therapeutic effect for drug like vardenafil, unless multiple doses are administered.

To achieve good permeation and/or absorption at the nasal sites, a low molecular weight (<1 kD) is preferable, with a good membrane partition coefficient (a good log P), a good aqueous solubility, and a desirable pKa that could lead to ionization and favorable permeation at the physiologic pH of the nose. Since the physiological pH of the nose is 6.4, a general recommendation is to keep pH of formulation between pH3.5-7.5 to avoid nasal membrane irritation (5, 10-11). However, to optimize individual drug solubility and permeability, it may be necessary to deviate from physiologic pH in the formulation. An envisaged target pH recommended is pH 3.5-7.5 (11)

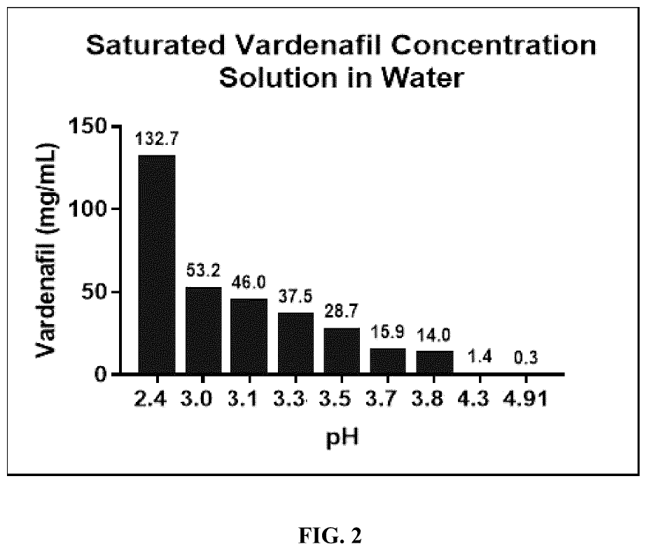

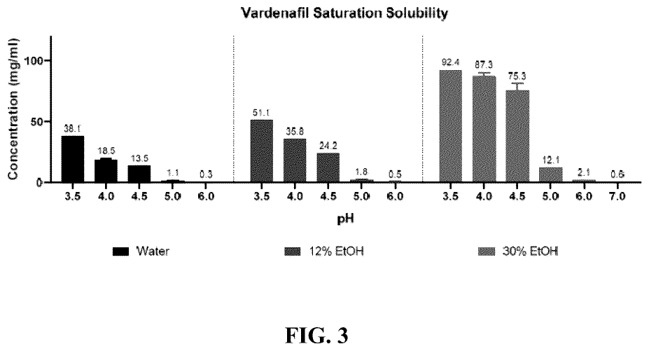

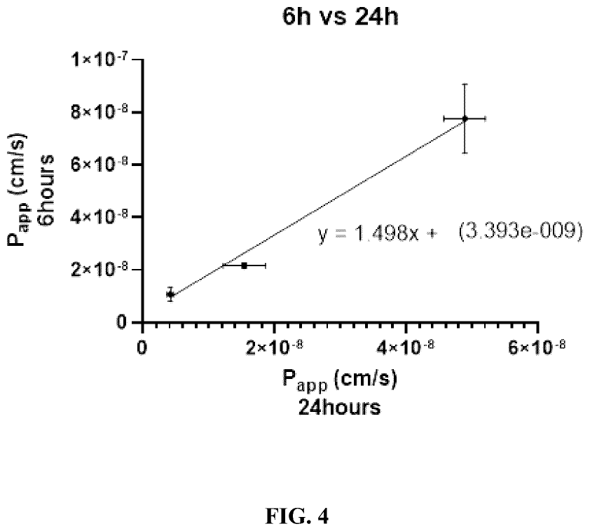

Vardenafil HCL trihydrate has a molecular weight of 579.1 (12). Vardenafil free base has a molecular weight of 488.6 and has a log P=3.64 and its pKa=7.15 (mostly basic) and 9.86 (mostly acidic). As a basic compound, vardenafil's aqueous solubility is pH dependent and reported to be less than 2 mg/ml at pH 4-7 (12-13). Although the solubility of vardenafil HCl trihydrate is higher than vardenafil base in water, the vardenafil API solubility still requires further improvement. (FIGS. 2-3). Since a drug must be in the soluble form for rapid nasal absorption to take place before exerting its therapeutic effect, thus achieving sufficient solubility and permeability of a desirable amount of the drug are the fundamental steps for vardenafil IN formulation. To optimize IN formulation for vardenafil API, determining its solubility and permeability experimentally at pH 3.5-7.5 will be required since there is no reliable/accurate method to predict drug solubility and permeability, especially for IN administration (14-15).

### Formulation Reagents

### Alcohols

In certain embodiments, the formulations and compositions for treating erectile dysfunction, increasing solubility of one of more phosphodiesterase inhibitor, and/or increasing permeability of one of more phosphodiesterase inhibitor described herein include at least one alcohol. In some embodiments, the formulations and compositions for treating erectile dysfunction, increasing solubility of one of more phosphodiesterase inhibitor, and/or increasing permeability of one of more phosphodiesterase inhibitor described herein include one or more weak salts. Exemplary weak salts include, for example, citric acid, tartaric acid, acetic acid, furmaric acid, lactic acid, ammonium chloride or similar organic salts, and others. In some embodiments, the formulations described herein comprise N-methyl pryrrolidone (NMP), Tween 80 or similar organic compounds. In some embodiments, the formulations described herein comprise a weak salt such as citric acid, tartaric acid, acetic acid, furmaric acid, lactic acid, ammonium chloride or similar organic salts, and others, or N-methyl pryrrolidone (NMP), Tween 80 or similar organic compounds in combination with one or more alcohol, a polyether, diethylene glycol monoethyl ether, a medium chain glyceride, one or more saturated polyglycolyzed C8-C10 glyceride, or a combination thereof. Alcohols are a family of compounds that contain one or more hydroxyl (—OH) group attached to a carbon atom of an alkyl group. An alcohol can have any number of carbon atoms in a chain. An alcohol can be a primary alcohol, a secondary alcohol, or a tertiary alcohol. Monohydric and polyhydric alcohols are known. Exemplary monohydric alcohols include methanol, ethanol, propanol, butanol, pentanol, hexanol, and others. Exemplary polyhydric alcohols include, for example, ethylene glycol, propylene glycol, glycerol (glycerin), and others. In some embodiments, the alcohol is ethanol. In some embodiments, the alcohol is present at a concentration of about 5%, about 6%, about 7%, about 8%, about 9%, about 10%, about 15%, about 20%, about 25%, about 30%, about 35%, about 40%, about 45%, about 50%, and any number or range in between. In some embodiments, the ethanol is present at a concentration of about 5% to about 40%. In some embodiments, the ethanol is present at a concentration of about 12%, about 25%, or about 30%. In some embodiments, the alcohol is glycerol (glycerin). In some embodiments, the glycerol is present at a concentration of about 1%, about 2%, about 3%, about 4%, about 5%, about 6%, about 7%, about 8%, about 9%, about 10%, about 15%, about 20%, about 25%, about 30%, about 35%, about 40%, about 45%, about 50%.

### Polyethers

The herein described formulations and compositions for treating erectile dysfunction, increasing solubility of one of more phosphodiesterase inhibitor, and/or increasing permeability of one of more phosphodiesterase inhibitor may, in certain embodiments, contain a polyether. Polyethers are polymers that contain more than one ether functional group. Polyethers include, for example, polyethylene glycol (PEG), polyethylene oxide (PEO), polyoxyethylene (POE), polypropylene glycol (PPG), polytetramethylene glycol (PTMG), polytetramethylene ether glycol (PTMEG), and paraformaldehyde. Aromatic polyethers include, for example, polyphenyl ether (PPE) and poly(p-phenylene oxide) (PPO). In some embodiments, the polyether is polyethylene glycol (PEG). The molecular weight of polyethylene glycol (PEG) may range from 300 g/mol to 10,000,000 g/mol. In some embodiments, the polyether is PEG 6000. In some embodiments, the polyethylene glycol (PEG) is present at a concentration of about 0.5%, about 1.0%, about 2.0%, about 3.0%, about 4.0%, about 5.0%, about 6.0%, about 7.0%, about 8.0%, about 9.0%, about 10%, about 11%, about 12%, about 13%, about 14%, about 15%, about 16%, about 17%, about 18%, about 19%, about 20%, about 25%, about 30%, and any number or range in between. In some embodiments, the polyethylene glycol (PEG) is present at a concentration of about 1% to about 20%. In some embodiments, the polyethylene glycol (PEG) is present at a concentration of about 5%.

### Glycerides

In certain embodiments, the formulations and compositions for treating erectile dysfunction, increasing solubility of one of more phosphodiesterase inhibitor, and/or increasing permeability of one of more phosphodiesterase inhibitor described herein include at least one or more glyceride. Glycerides are esters formed from glycerol and fatty acids. Exemplary glycerides include mono-, di-, and triglycerides. In some embodiments, the formulations and compositions described herein contain medium chain glycerides. In some embodiments, the formulations and compositions described herein contain polyglycolyzed C8-C10 glycerides. In some embodiments, the polyglycolyzed C8-C10 glyceride is a saturated polyglycolyzed C8-C10 glyceride. In some embodiments, the formulations and compositions described herein comprise a mixture of glycerides. Glycerides in a mixture can be unsaturated or saturated. In some embodiments, the mixture of glycerides comprises additional chemicals or compounds. In some embodiments, the glycerides comprise polyoxylglycerides. In some embodiments, the glycerides comprise caprylocaproyl polyoxyl-8 glycerides or caprylocaproyl macrogol-8 glycerides. In some embodiments, the glycerides comprise caprylic/capric glycerides. In some embodiments, caprylic/capric glycerides further comprise a polyethylene glycol, such as PEG-8, for example. In some embodiments, the formulations and compositions described herein comprise LABRASOL.

### Solvent Stabilizer/Penetration Enhancer

In some embodiments, the formulations and compositions for treating erectile dysfunction, increasing solubility of one of more phosphodiesterase inhibitor, and/or increasing permeability of one of more phosphodiesterase inhibitor described herein may contain certain other compounds or chemicals that serve as solvents, stabilizers or penetration enhancers. As an example, the formulations and compositions described herein may contain diethylene glycol monoethyl ether. Diethylene glycol monoethyl ether is also known as 2-(2-Ethoxyethoxy)ethanol and is sold under the brand name TRANSCUTOL. This compound can serve as a high purity solvent and stabilizer and is associated with skin penetration enhancement in topical dosage forms. Other suitable solvents, stabilizers and penetration enhancers will be well-known to those having ordinary skill in the art. The amount of such compounds can vary according to the formulation desired, such as in the range from 0.1 to 99.9% by weight, from 1.0 to 99% by weight, from 5% to 95% by weight, from 10% to 90% by weight, or from 20% to 80% by weight.

### Buffering Agents

Buffering Agents that can be used in the embodiments described herein will be known to those skilled in the art. Please see “Handbooks Pharmaceutical Excipients (Second Edition), edited by Ainley Wade and Paul J W Weller, The Pharmaceutical Press London, 1994,” which is incorporated herein by reference. Exemplified buffering agents include, but are not limited to, phosphates, such as sodium phosphate; phosphates monobasic, such as sodium dihydrogen phosphate and potassium dihydrogen phosphate; phosphates dibasic, such as disodium hydrogen phosphate and dipotassium hydrogen phosphate; citrates, such as sodium citrate (anhydrous or dehydrate); bicarbonates, such as sodium bicarbonate and potassium bicarbonate. The amount of buffering agents used in the formulations and methods described herein is readily determined by those skilled in the art, which depend on preferable pH values. Certain embodiments contemplated herein feature a formulation or composition having a pH of about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 8.0, about 7.6, about 7.7, about 7.8, about 7.9, about 8.0, about 8.1, about 8.2, about 8.3, about 8.4, about 8.5, about 8.6, about 8.7, about 8.8, about 8.9 or about 9.0, and any number or range in between. In some embodiments, the formulation or composition has a pH of about 3.5 to about 8.0. In some embodiments, the formulation or composition has a pH of about 3.5 to about 6.5. In some embodiments, the formulation or composition has a pH of about 4.0 to about 5.0.

### Carriers

The carrier suitably used in the embodiments described herein depends on the specific formulation or composition of the medicament. The carriers include, without limitation, fillers, binders, lubricants, diluents, sweetening and flavoring agents, preservatives, disintegrators, grilling agents, permeation enhancers. Examples of the carriers include starch, gelatin, natural sugars, corn, natural and synthetic gums such as acacia, sodium alginate, methylcellulose, carboxymethylcellulose, polyethylene glycol, waxes, boric acid, sodium benzoate, sodium acetate, sodium chloride, agar, bentonite, agar gum, stearates such as sodium stearate, HPMC, palmitic acid, dimethyl sulfoxide, N,N-dimethyl acetamide, N,N-dimethylformamide, 2-pyrrolidone, 1-methyl-2-pyrrolidone, 1,5-dimethyl-2-pyrrolidone, 1-ethyl-2-pyrrolidone, 2-pyrrolidone-5-carboxylic acid, N,N-dimethyl-m-toluamide, urea, ethyl acetate, 1-dodecylazacycloheptan-2-one (Azone®), oleic acid, ethylene vinylacetate copolymer, polyvinyl chloride, polyethylene, polydiethyl phthalate.

### GENERAL DESCRIPTION OF VARIOUS EMBODIMENTS

### Exemplary Formulations for Enhancing Solubility/Permeation of One or More Phosphodiesterase Inhibitor Across a Mucosal Membrane

Described herein, in some embodiments, are formulations for enhancing solubility/permeation of one or more phosphodiesterase inhibitor across a mucosal membrane, comprising: (a) one or more phosphodiesterase inhibitor; and (b) an organic-aqueous solvent comprising an alcohol, a glycol, diethylene glycol monoethyl ether, a medium chain glyceride, one or more saturated polyglycolyzed C8-C10 glyceride, or a combination thereof; wherein the formulation has a pH of about 3.5 to about 8.0 and wherein the organic-aqueous solvent enhances solubility of the one or more phosphodiesterase inhibitor relative to solubility of the one or more phosphodiesterase inhibitor in water. In some embodiments, the organic-aqueous solvent comprises an alcohol. In some embodiments, the alcohol is ethanol or glycerol. In some embodiments, the ethanol is present at a concentration of 5% to 40%. In some embodiments, the ethanol is present at a concentration of 12%, 25%, or 30%. In some embodiments, the organic-aqueous solvent comprises a polyether. In some embodiments, the polyether is polyethylene glycol. In some embodiments, the polyethylene glycol is PEG 6000 or PEG 400. In some embodiments, the polyethylene glycol is present at a concentration of 1% to 20%. In some embodiments, the polyethylene glycol is present at a concentration of 5%. In some embodiments, the formulation described herein comprises a weak salt. Non-limiting examples of a weak salt include citric acid, tartaric acid, acetic acid, furmaric acid, lactic acid, ammonium chloride, similar organic salts, N-methyl pryrrolidone (NMP), Tween 80, or similar organic compounds. In some embodiments, the formulation described herein comprises one or more alcohol, a polyether, diethylene glycol monoethyl ether, a medium chain glyceride, one or more saturated polyglycolyzed C8-C10 glyceride, or a combination thereof. In some embodiments, the formulation has a pH of about 3.5 to about 8.0. In some embodiments, the formulation has a pH of about 3.5 to about 5.0. In some embodiments, the phosphodiesterase inhibitor is vardenafil, sildenafil, tadalafil, or a combination thereof. In some embodiments, the phosphodiesterase inhibitor is vardenafil. In some embodiments, the phosphodiesterase inhibitor is sildenafil. In some embodiments, the phosphodiesterase inhibitor is tadalafil. In some embodiments, the phosphodiesterase inhibitor is vardenafil in combination with sildenafil and/or tadalafil.

### Exemplary Methods for Treating Erectile Dysfunction

Described herein, in some embodiments, are methods of treating erectile dysfunction of a subject in need thereof, comprising contacting a mucosal membrane of the subject with a formulation disclosed herein, thereby treating the erectile dysfunction of the subject. In some embodiments, contacting the mucosal membrane comprises intranasal administration. In some embodiments, contacting the mucosal membrane comprises sublingual administration.

### Exemplary Methods of Preparing Formulations for Treating Erectile Dysfunction

Described herein, in some embodiments, are methods of preparing a formulation for treating erectile dysfunction of a subject, comprising: (a) adding one or more phosphodiesterase inhibitor to an organic-aqueous solvent comprising an alcohol, a polyether, diethylene glycol monoethyl ether, a medium chain glyceride, one or more saturated polyglycolyzed C8-C10 glyceride, or a combination thereof; (b) adjusting the pH of the organic-aqueous solvent comprising the one or more phosphodiesterase inhibitor to about 3.5 to about 8.0; wherein treating the erectile dysfunction comprises contacting a mucosal membrane of the subject with the formulation. In some embodiments, solubility of the one or more phosphodiesterase inhibitor is increased in the organic-aqueous solvent relative to solubility of the one or more phosphodiesterase inhibitor in water. In some embodiments, permeability of the one or more phosphodiesterase inhibitor across the mucosal membrane is increased in the organic-aqueous solvent relative to permeability of the one or more phosphodiesterase inhibitor in water. In some embodiments, bioavailability of the one or more phosphodiesterase inhibitor is increased in the organic-aqueous solvent relative to bioavailability of the one or more phosphodiesterase inhibitor in water. In some embodiments, the organic-aqueous solvent comprises an alcohol. In some embodiments, the alcohol is ethanol or glycerol. In some embodiments, the ethanol is present at a concentration of 5% to 40%. In some embodiments, the ethanol is present at a concentration of 12%, 25%, or 30%. In some embodiments, the organic-aqueous solvent comprises a polyether.

In some embodiments, the polyether is polyethylene glycol. In some embodiments, the polyethylene glycol is PEG 6000 or PEG 400. In some embodiments, the polyethylene glycol is present at a concentration of 1% to 20%. In some embodiments, the polyethylene glycol is present at a concentration of 5%. In some embodiments, the formulation described herein comprises a weak salt. Non-limiting examples of a weak salt include citric acid, tartaric acid, acetic acid, furmaric acid, lactic acid, ammonium chloride, similar organic salts, N-methyl pryrrolidone (NMP), Tween 80, or similar organic compounds. In some embodiments, the formulation described herein comprises one or more alcohol, a polyether, diethylene glycol monoethyl ether, a medium chain glyceride, one or more saturated polyglycolyzed C8-C10 glyceride, or a combination thereof. In some embodiments, the formulation has a pH of about 3.5 to about 8.0. In some embodiments, the formulation has a pH of about 3.5 to about 5.0. In some embodiments, the phosphodiesterase inhibitor is vardenafil, sildenafil, tadalafil, or a combination thereof. In some embodiments, the phosphodiesterase inhibitor is vardenafil. In some embodiments, the phosphodiesterase inhibitor is sildenafil. In some embodiments, the phosphodiesterase inhibitor is tadalafil.

In some embodiments, the formulations described herein comprise an organic-aqueous solvent comprising more than one organic solvent or component. Exemplary organic solvent or component mixtures include, for example, PEG and ethanol in water. In some embodiments, the PEG in an aqueous organic solvent mixture is PEG 400. In some embodiments, the ethanol is present in the aqueous organic solvent mixture at a concentration of about 5% to about 40%. In some embodiments, the ethanol is present in the aqueous organic solvent mixture at a concentration of about 12%. In some embodiments, the PEG is present in the aqueous organic solvent mixture at a concentration of about 1% to about 40%. In some embodiments, the PEG 400 is present in the aqueous organic solvent mixture at a concentration of about 1% to about 40%. In some embodiments, the PEG 400 is present in the aqueous organic solvent mixture at a concentration of about 10%, about 15%, or about 20%. In some embodiments, the formulation comprises 10% PEG 400 in 12% ethanol. In some embodiments, the formulation comprises 15% PEG 400 in 12% ethanol. In some embodiments, the formulation comprises 20% PEG 400 in 12% ethanol. Formulations comprising more than one organic solvent or component in water can be used in any of the methods described herein.

In some embodiments, wherein the organic-aqueous solvent comprises more than one organic solvent or component, the second organic component is chosen for the purpose of enhancing at least one of property selected from the group solubility, stability, permeability, and safety. In some embodiments, the formulation has a pH of about 3.5 to about 8.0.

While many of the examples disclosed herein relate to formulations used in treating erectile dysfunction, it will be understood that the formulation may be combined with one or more of any ionizable drug, and consequently it is envisioned that this formulation would have use in the treatment of any disease, disorder, or symptom that is targeted by the one or more ionizable drug.

### Example Method of Formulation

As disclosed herein, the new mucosal formulation (eg. IN, sublingual formulation) serves to achieve (a) a good therapeutic effect or effective drug concentration in the body (e.g. plasma drug concentration) as well as (b) rapid onset of effect or fast peak drug concentration in the body (Tmax) as compared to that from an established oral route of administration, or achieve a similar effect as that from an established injectable route of administration (e.g., intravenous, or subcutaneous route). In some alternatives, the formulation is optimized for a given ionizable compound using the following methodology:

### Step 1: Estimating an Effective Drug Dose of the Mucosal Formulation

The goal of estimating the effect drug dose is so that it will achieve an equivalent effect or area-under-the-concentration-time curve (AUC) as that from oral or injectable route. In some alternatives, this comprises an initial review of the bioavailability from oral dosing of the established oral drug or bioavailability from injectable dosing of the injectable drug, to calculate the effective mucosal dose. For example, if the drug X has an oral bioavailability of 0.5 compared to intravenous dosing due to liver first-pass metabolism. The IN route will avoid liver first-pass metabolism and its IN dose can be estimated to be half of the oral dose. Thus if the oral dose of drug X is 10 mg, its IN dose can be estimated to be about 5 mg.

### Step 2: Estimating Minimum Soluble Drug Concentration or Solₘᵢₙ Required for Mucosal Drug Administration.

Certain mucosal administration have a limited volume that can be administered to the site. For example, for IN administration, the optimal volume by nasal spray is no more than 0.2 ml usually 0.1-0.15 ml per nostril to prevent “dripping out”. Therefore, the solution volume administered to both nostrils is about 0.2-0.3 ml. Hence the Solmin required for the example of the above IN 5 mg drug X (from step 1) is estimated as 5 mg/0.2 ml or 5 mg/0.3 ml=25 mgml or 17 mg/ml respectively when using 0.1 ml spray or 0.15 ml/spray to each of 2 nostrils. Similarly, for sublingual spray, using the selected desired volume, such Solmin can be similarly calculated.

### Step 3: Determine the Reference Drug Flux or Jss_{(Ref)}

The drug flux or Jss_{(ref)}, is defined as the reference amount of drug per time per area permeated across the mucosal membrane from a drug in aqueous solution administered to the membrane at a particular pH. It is well known the drug flux or Jss is composed of solubility times apparent permeability (Papp), and Papp of drug across a biological membrane is primarily dependent on the unstirred water layer (UWL) and the permeability properties of the membrane for the drug. The UWL of the mucosal membrane (such as nasal membrane) is covered with mucus, a gel-like fluid mainly composed of water. In view of this structure, the present patent hypothesizes that the Jss of the drug in pure aqueous solution can serve as a reference for Jss of the drug in organic-aqueous solution (which is the new formulation of the drug intended for mucosal administration. Since Jss=(Papp)(solubility), the Papp can be determined by the artificial membrane, PAMPA (which has both sides of the membrane in contact with UWL)(16-19). Papp from PAMPA can be a useful approach in the estimation of Jss_{(ref} which is proposed to serve as a guide for organic-aqueous formulation of the drug. Thus, the current patent further proposes:

JSS_{(ref)}=(JSS_{pHmax})(Solₘᵢₙ/Drug_{(ssol)pHmax})

where for an ionizable drug in aqueous saturated solution, JSs_{pHmax} is the maximum value of Jss from Jss vs pH plot determined by PAMPA, the pH corresponding to the maximum Jss is the pHmax, Solₘᵢₙ has been defined in Step 2, and Drug_{(ssol)pHmax} is the saturated solubility of the drug at the pHmax. The Drug_{(ssol)pHmax} and Papp are usually determined at room temperature and atmospheric pressure.

### Step 4: Determining Jss_{pHmax} of the Drug in Organic-Aqueous Solution

Drug_{(ssol)pHmax} and Papp at pHmax or Papp_{pHmax} for the drug in the organic-aqueous solution are screened using the usual “shake flask” method (determination of solubility)(20-22) and PAMPA (determination of permeability) respectively. If the Drug_{(ssol)pHmax} is ≥ to Solₘᵢₙ (as define in Step 2) and the combined (Drug_{(ssol)pHmax)}(Papp_{pHmax}) or Jss_{PHmax} for the drug in a given organic-aqueous solution (or the specific formulation for mucosal administration) equals or exceeds that of Jss_{(ref)} then that specific formulation can qualify as a suitable mucosal formulation of the drug to be administered (e.g. by IN or sublingual route) capable of achieving an equivalent therapeutic effect as well as a shorter Tmax as that from the oral administration, as calculated in Step 1.

### Step 5: Conformation of Jss_{PHmax} Using an Appropriate Cell Line Model

Although Papp obtained by PAMPA is relatively simple and easy, a more physiologic approach of determining Papp of the drug in aqueous and various organic-aqueous solutions will provide further confirmation of the chosen formulation described in Steps 1-4 above. For IN formulation, appropriate Papp confirmation determined using Calu-3 cell line model can be used(23-24). For sublingual formulation, a HO-1U-1 cell line can be used (25).Any organic-aqueous formulation that results in significantly lower Papp as compared to that in aqueous solution at the pHmax, should be excluded.

### EXAMPLES

Additional embodiments are disclosed in further detail relating to the identification of specific formulations and methods to achieve effective IN or sublingual dosing of phosphodiesterace inhibitors. These are not in any way intended to limit the scope of the claims. Furthermore, the identification of specific formulations and methods to achieve effective IN or sublingual dosing described herein can be applicable to any other ionizable compounds. Included in the following are specific Examples A1, A2, B1, B2, and D1-D4 which represent sequential steps of method of identifying desirable IN formulations based on combined solubility and permeability profile as well as dosing required of vardenafil and sildenafil to achieve rapid and effective concentration in the body. Specific Examples of C1, C2, C3 and D5 are in vivo evidence of confirming the appropriateness of formulations and method of IN dosing identified by the above stepwise method.

### Example A1

### Intranasal (IN) Dosing and Formulation

Embodiments of the minimal IN effective dosing requirement of the phosphodiesterase inhibitor, e.g. vardenafil/sildenafil required to achieve a therapeutic effect equivalent to an approved oral dosing described herein was estimated using specific calculations. Such calculation assumes that sufficient vardenafil/sildenafil and nasal permeability can be identified and can lead to desired plasma concentration and bioavailability.

The desired dosing further requires an IN formulation of vardenafil API or sildenafil API to provide an amount of vardenafil HCl trihydrate that will achieve a similar but significantly earlier effective concentration as that from the oral route. For example, if 10-20 mg oral vardenafil dose is approved by FDA, an IN dosing with a suitable formulation should lead to achieving similar bioavailability but a much earlier peak time (Tmax) as that from the oral route. Since IN dose is administered either via a nasal spray or nose drop, the amount of IN dose can be estimated from the volume of vardenafil formulation solution administered intranasally times its concentration after adjustment for relative bioavailability. An example of the calculation is provided below.

As IN administration normally uses a volume of 50-200 ul/spray to each nostril (a spray volume larger than 200 ul will most likely result in some volume dripping out of nose), thus a desirable spray volume can be set at 100 ul/nostril or 2×100 ul for 2 nostrils as the IN dose to be equivalent to 10 mg vardenafil oral dose. Doubling the volume of nasal spray should provide IN dose equivalent to 20 mg oral dose. Other phosphodiesterase inhibitors such as tadalafil or sildenafil IN volume/dose can be similarly calculated to equal to its oral dose.

Assuming absorption of vardenafil aqueous solution via oral administration is related to its IN administration of a suitable formulation, and based on the known bioavailability/pharmacokinetics of vardenafil from its oral administration, its intranasal administration can be assumed to yield a bioavailability equivalent to 0.4-0.8 oral dose, but can achieve a much earlier peak concentration. This is based on the known advantage of IN administration which can avoid first-pass liver metabolism and achieving rapid permeation via nasal membrane, similar as the bioavailability from pulmonary inhalation published previously (26). This will require a minimum 2 mg/100 ul concentration or a minimum solubility of 20 mg/ml vardenafil HCl trihydrate for the IN formulation. Other phosphodiesterase inhibitors, such as sildenafil or tadalafil can be calculated using a similar approach. A special device can be used for intranasal administration and can be delivered to each of two nostrils. Devices for intranasal administration are commercially available from Aptar, for example.

### Example A2

### Screening for Solubility and Stability Characteristics of Vardenafil in Organic-Aqueous Mixtures

Embodiments of vardenafil solubility and stability described herein was determined in various organic-aqueous mixtures.

The active pharmaceutical ingredient, vardenafil HCl trihydrate, has a molecular weight of 579.1 g/mole with corresponding free base of 488.6 g/mole (12). The vardenafil base has a pka=7.15 and 9.86, and log P=3.64. Vardenafil solubility is about 8.8 g/L at pH 1, 3 g/L at pH 2, 1.6 g/L at pH 3, 0.88 g/L at pH 4, 0.16 g/L at pH 5 and 0.019 g/L at pH 6 (13). The solubility of the active pharmaceutical ingredient (API), vardenafil HCl trihydrate, in water is much better (FIG. 3). Such solubility however is still low and its decreasing aqueous solubility with increasing pH is a significant obstacle to achieving rapid and sufficient permeation and/or absorption via sublingual and intranasal administration. Thus, despite an excellent membrane partition coefficient (log P=3.64) of vardenafil base, sublingual or intranasal administration using aqueous vardenafil solution cannot yield a sufficient bioavailability compared to oral administration when administered close to or at the suitable physiologic pH at the nasal or sublingual sites(see calculation in EXAMPLE A1).

Vardenafil API, however, can achieve an improved solubility in certain solvents, e.g. alcohol (13) or other organic-aqueous mixture solvents. The use of pure alcoholic solutions of vardenafil is a concern due to potential membrane irritation and damage. Thus, an alcoholic-aqueous mixture or other organic-aqueous mixture that is relatively safe or well tolerated by human subjects, such as those organic compounds (at relatively low concentrations) under the “generally regarded as safe” or “GRAS” category is preferable. For such reasons, the use of a 12% alcohol solvent in nasal products is recognized by the FDA as a tolerable concentration for human subjects (27,28). Although a 12% alcohol can rapidly solubilize vardenafil API, it will precipitate within 24 h. Thus a “shake flask” method over 3 days was utilized to determine saturated solubility (20-22).

As an example, the solubility of saturated vardenafil in water at different pH was first screened, followed by screening the permeability at different pH. Such information will be used for generating the optimal combined solubility and permeability (i.e. Jss) in the aqueous system which will be used to provide an initial clue of desirable pH, solubility and permeability for the organic-aqueous solutions to be used as the desired suitable IN vardenafil formulation and dose.

In addition, any mixture of solvents must be capable of solubilizing vardenafil for rapid and sufficient absorption when administered sublingually or intranasally.

As an example, the solubility of vardenafil API in ethanol-aqueous mixtures was screened first, followed by screening the permeability at different pH to determine the optimal solubility and permeability that can be suitable for sublingual and intranasal administration.

### Materials

Vardenafil Hydrochloride (CAS No. 224785-91-5) was purchased from India Alembic Pharmaceutical Ltd, Gujarat-391450 India (Lot #1704002361).

Tadalafil (TAD) 5 mg tablets were purchased from Polpharma (Poland).

Acetonitrile ≥99.5% ACS (CAS No. 75-05-8) was purchased from VWR Chemicals BDH®.

Methanol (“MeOH”) was purchased from VWR Chemicals BDH®.

Ethanol 190-Proof (CAS No. 64-17-5) was purchased from EMD Millipore (Burlington, MA, USA).

Syringe Filter w/0.2 μm pore size Cellulose Acetate Membrane (Cat #28145-475) was purchased from VWR (Radnor, PA, USA).

Polyethylene glycol 400 (Lot 52081314) was purchased from EMD Millipore (Burlington, MA, USA).

Glycerin or glycerol (Lot 70K0044) was purchased from Sigma-Aldrich (St. Louis, MO, USA).

Calcium Lactate Pentahydrate (Lot SLCB7173) was purchased from Sigma-Aldrich (St. Louis, MO, USA).

Glacial Acetic Acid (Lot B21R026) was purchased from Alfa Aesar (Haverhill, MA, USA).

NMP (1-Methyl-2-Pyrrolidinone) (Lot 51K3683) was purchased from Sigma-Aldrich (St. Louis, MO, USA).

### Equipment

Accumet Basic pH meter was purchased from Fisher Scientific (Leicestershire, UK).

Agilent 1260 Infinity HPLC system which consisted of a G1311B 1269 Quat Pump, a G7129 1260 vial sampler, and a G1315D 1260 DAD VAL detector was purchased from Agilent (Santa Clara, CA).

Analytical Balance was purchased from Mettler-Toledo, LLC (Columbus, OH).

### Procedure

For screening of various organic solvents, the solubility of vardenafil API in different % ethanol-aqueous mixtures was first investigated and compared to solubility in pure water. As disclosed herein, the solutions were prepared by the “shake flask” method. Briefly, increasing amounts of vardenafil API were added to different mixtures until saturation. The saturated organic-aqueous mixtures were adjusted for pH (at the range pH 3.5-7.5) with the use of a pH meter. The saturated solution was shaken slowly with a magnetic stirrer at room temperature or shaken rapidly several times a day for 24 hours or longer, up to 3 days. Afterwards, the solutions were filtered using the VWR 0.2 micron filter. The filtrate was then used for inspection of clarity and concentration was determined by HPLC. In addition, the saturated solubility of vardenafil various organic-aqueous solution, e.g., in glycerin (glycerol), polyethylene glycol 400 (PEG) and combination of two organic solvents were investigated. Vardenafil solubility in some other representative organic-aqueous solutions were also investigated.

### (a) HPLC Method for Vardenafil Concentration Determination

For solubility studies as well as concentration determination from permeability studies (see Example B1 and B2), the samples were prepared for HPLC analysis by mixing 1:1 with MeOH. The standard curves were prepared in MeOH and mixed with PBS so that the analytical matrices were the same. The HPLC analysis was run through an Agilent 1260 Hypersil BDS-C8, 5 μm, 4.0×250 mm column (PN 79926B8-584, SN USUE000480, LN 512010961) using Acetonitrile:0.02M Sodium Phosphate Buffer pH 4 (35:65 v/v) isocratic flow at 1 mL/min for 10 minutes.

### Results

The validity of the assay was assessed according to FDA guidance with regard to linearity, sensitivity, repeatability, stability, precision, and accuracy. The calibration curve of vardenafil was linear over the concentration range of 0.2-200 ug/ml. The correlation coefficient (r²) was greater than 0.99 for each of 3 different runs. For quality control samples of 0.5, 10 and 200 ug/ml, the relative standard deviation (RSD) values for precision were 1.8 to 6.1% (interday) and 0.07 to 4.1% (intraday). The accuracy (% bias) ranged −4.2% to 2.2% (interday) and −0.9 to 3.4% (intraday). The lower limit of quantitation was 0.2 ug/ml.

Table 1 shows a comparison of saturated solubility of vardenafil concentration in several organic-aqueous solvents at different pH. Table 2 shows the inter-day accuracy and precision of solutions containing 6 different solutions and 2 different pH values measured using standard curve for each solution and then compared with 50% methanol standard curve. Table 3 shows a comparison of saturated solubility of vardenafil API at pH 4.0 in different solvents.

TABLE 1
Comparison of saturated solubility in
the indicated organic-aqueous solvent

| | pH | | | | | |
|---|---|---|---|---|---|---|
| Sample Solutions | 3.5 | 4.0 | 4.5 | 5.0 | 6.0 | |
| 10% Glycerin in H₂O | 32.06 | 16.84 | 1.44 | 0.43 | 0.10 | mg/mL |
| 20% Glycerin in H₂O | 31.42 | 18.44 | 1.67 | 0.27 | 0.10 | mg/mL |
| 10% PEG400 in H₂O | 34.30 | 20.37 | 2.46 | 0.56 | 0.17 | mg/mL |
| 15% PEG400 in H₂O | 32.79 | 18.23 | 3.56 | 1.01 | 0.86 | mg/mL |
| 20% PEG400 in H₂O | 30.67 | 20.98 | 2.59 | 1.49 | 0.44 | mg/mL |
| 10% PEG400 in 12% | 48.91 | 40.73 | 8.11 | 1.51 | 0.28 | mg/mL |
| EtOH | | | | | | |
| 15% PEG400 in 12% | 46.54 | 44.23 | 9.89 | 2.87 | 0.38 | mg/mL |
| EtOH | | | | | | |
| 20% PEG400 in 12% | 47.06 | 45.56 | 11.50 | 2.90 | 0.41 | mg/mL |
| EtOH | | | | | | |
| 12% EtOH | 51.28 | 47.18 | 22.61 | 1.06 | 0.04 | mg/mL |

Abbreviation: PEG = polyethylene glycol

TABLE 2
Comparison of dilution accuracy and bias in analysis
of the indicated organic-aqueous solvent

| Solution | Slope | Intercept | R² | Accuracy (%) | % Bias |
|---|---|---|---|---|---|
| 50% MeOH | 0.03881 | 8.12E−04 | 0.999 | | |
| pH 2 in 50% MeOH | 0.03870 | −1.27E−03 | 0.998 | 100% | 0% |
| pH 12 in 50% MeOH | 0.03894 | 7.09E−05 | 0.992 | 101% | 1% |
| EtOH (12%) | 0.03695 | 4.97E−03 | 0.984 | 95% | −5% |
| PEG400 (15%) | 0.04009 | −4.32E−03 | 0.991 | 108% | 8% |
| NMP (20%) | 0.04241 | 2.06E−03 | 0.974 | 106% | 6% |
| Calcium Lactate (5%) | 0.03871 | 3.29E−03 | 0.990 | 91% | −9% |
| EtOH/PEG400 (12%/15%) | 0.03901 | −5.09E−04 | 0.993 | 101% | 1% |
| Acetic Acid (2%) | 0.03649 | −4.39E−04 | 0.996 | 94% | 6% |

The dilution accuracy was determined by comparing various solutions at 20 mg/ml. The RSD was 3.1%. The stability of the quality control samples was tested by re-injecting the samples at 0, 9, 18 and 24 h after reconstitution and storage in an autosampler at 200 C. The RSD values ranged from 2.8-7.8%.

TABLE 3
Vardenafil API solubility at pH 4 in different solvents*

| | | Average of | |
|---|---|---|---|
| | | Sat Conc | SD |
| Row Labels | N | (mg/mL) | (mg/mL) |
| Water | 16 | 18.19 | 4.36 |
| Acetic Acid (1%) | 1 | 11.60 | |
| Acetic Acid (10%) | 1 | 79.12 | |
| Acetic Acid (20%) | 1 | 141.70 | |
| Acetic Acid (5%) | 1 | 52.01 | |
| Calcium Lactate (1%) | 2 | 8.49 | 0.87 |
| Calcium Lactate (3%) | 2 | 16.36 | 2.50 |
| Calcium Lactate (5%) | 6 | 14.26 | 0.79 |
| EtOH (3%) | 1 | 28.07 | |
| EtOH (5%) | 5 | 24.98 | 3.80 |
| EtOH (8%) | 4 | 29.95 | 2.29 |
| EtOH (10%) | 5 | 31.71 | 0.87 |
| EtOH (12%) | 7 | 31.24 | 8.67 |
| EtOH (15%) | 1 | 37.68 | |
| EtOH (20%) | 5 | 42.41 | 6.72 |
| EtOH (30%) | 5 | 54.13 | 22.91 |
| EtOH (35%) | 1 | 101.59 | |
| NMP (5%) | 4 | 37.38 | 1.21 |
| NMP (10%) | 12 | 53.19 | 6.43 |
| NMP (15%) | 4 | 52.98 | 4.35 |
| NMP (20%) | 4 | 56.78 | 3.06 |
| NMP (25%) | 13 | 88.35 | 27.24 |
| NMP (35%) | 1 | 107.49 | |
| NMP (40%) | 3 | 127.81 | 15.41 |
| PEG400 (10%) | 14 | 20.25 | 4.31 |
| PEG400 (15%) | 12 | 17.05 | 3.94 |
| PEG400 (20%) | 16 | 20.06 | 5.16 |
| PEG400 (40%) | 3 | 20.01 | 4.98 |
| PEG400 (5%) | 11 | 16.52 | 4.33 |
| Calcium Lactate/NMP | 2 | 31.95 | 4.82 |
| (3%/10%) | | | |
| Calcium Lactate/NMP | 1 | 29.11 | |
| (5%/10%) | | | |
| EtOH/Calcium Lactate | 2 | 18.79 | 1.00 |
| (10%/5%) | | | |
| EtOH/Calcium Lactate | 5 | 23.14 | 2.61 |
| (12%/5%) | | | |
| EtOH/Calcium Lactate | 1 | 16.56 | |
| (5%/5%) | | | |
| EtOH/Calcium Lactate | 1 | 16.95 | |
| (8%/5%) | | | |
| EtOH/NMP (5%/5%) | 3 | 37.91 | 3.11 |
| EtOH/NMP (5%/10%) | 5 | 51.80 | 11.20 |
| EtOH/NMP (5%/15%) | 3 | 46.38 | 3.58 |
| EtOH/NMP (8%/5%) | 3 | 37.76 | 2.92 |
| EtOH/NMP (8%/10%) | 3 | 46.90 | 3.64 |
| EtOH/NMP (8%/15%) | 1 | 56.05 | |
| EtOH/NMP (10%/5%) | 3 | 40.06 | 3.38 |
| EtOH/NMP (10%/10%) | 4 | 52.74 | 2.69 |
| EtOH/NMP (10%/15%) | 3 | 56.84 | 1.77 |
| EtOH/NMP (12%/10%) | 1 | 70..47 | |
| EtOH/NMP (12%/15%) | 2 | 80.81 | 2.82 |
| EtOH/PEG400 (3%/10%) | 1 | 17.72 | |
| EtOH/PEG400 (5%/5%) | 1 | 18.68 | |
| EtOH/PEG400 (5%/10%) | 2 | 27.45 | 15.75 |
| EtOH/PEG400 (8%/10%) | 5 | 27.03 | 10.78 |
| EtOH/PEG400 (8%/15%) | 3 | 24.90 | 0.87 |
| EtOH/PEG400 (8%/20%) | 3 | 23.99 | 1.37 |
| EtOH/PEG400 (10%/10%) | 3 | 25.73 | 1.75 |
| EtOH/PEG400 (10%/15%) | 3 | 24.90 | 2.37 |
| EtOH/PEG400 (10%/20%) | 3 | 24.99 | 1.35 |
| EtOH/PEG400 (12%/10%) | 4 | 26.37 | 3.96 |
| EtOH/PEG400 (12%/15%) | 6 | 28.60 | 5.94 |
| EtOH/PEG400 (12%/20%) | 3 | 29.57 | 1.35 |
| EtOH/PEG400 (15%/10%) | 1 | 23.20 | |
| EtOH/PEG400 (15%/15%) | 1 | 20.52 | |
| PEG400/NMP (15%/10%) | 1 | 41.59 | |

*Determined at room temperature at mean pH 4.0 (3.9-4.1), Sat Conc—saturated concentrations, SD—standard deviation

### Conclusion

As disclosed herein, organic-aqueous mixtures, such as an ethanol-aqueous mixture, can significantly enhance vardenafil solubility as compared to solubility in a pure aqueous solution (FIGS. 3 and Table 3). The solubility of vardenafil API is pH-dependent, with higher solubility at lower pH. The organic-aqueous mixtures, such as an ethanol-aqueous mixture, can significantly enhance vardenafil solubility as compared to solubility in water. Vardenafil solubility can be further enhanced by increasing the % organic solvent concentration, such as ethanol, for example. Furthermore, combination of certain organic solution mixtures can improve solubility of vardenafil, e.g. 15% PEG-12% EtOH-aqueous mixture when compared to PEG15-aqueous solution.

The results disclosed herein indicate that using the methanol standard curve for assay can produce accurate and precise vardenafil concentration determination in different solvents or solvent mixtures as well as at different pH. The dilution of 100-fold from 20 mg/ml concentration can also be accurately/precisely measured within +/−15%.

If a minimum solubility of vardenafil API 20 mg/ml at pH4 is desired for IN formulation, water which has a mean saturated solubility below 20 mg/ml at pH4 is not a suitable solvent for vardenafil IN formulation. However, a number of other organic-aqueous solvents under GRAS category with higher solubility of 20 mg/ml at pH4 have been identified (see Table 3) and further mixture of these solutions are likely to be suitable for vardenafil IN formulation, as disclosed herein.

### Example B1

### Screening for Permeability and Flux Using PAMPA for Selection of Vardenafil IN Formulation

This example describes the determination of permeability of vardenafil using a parallel artificial membrane permeability assay (PAMPA).

As disclosed herein, the permeability of vardenafil API in different solvents at room temperature and atmospheric pressure was screened using in vitro PAMPA. The PAMPA predicts passive absorption of drugs and is suitable for studies with many solutions including ethanol (up to concentration of 30%)(22-24). The unit of measurement is the apparent permeability (Papp) obtained at steady state, expressed as cm/second. Also another associated measurement is the maximum flux (Jss) at a particular pH, expressed as the quantity of drug across the unit area per sec, is calculated from the Papp and saturated solubility.

As disclosed herein, the effect of an ethanol-aqueous solution on improving permeability/absorption of the drug across a mucosal membrane was determined. Given that a 12% ethanol-aqueous solution and a 30% ethanol-aqueous solution can improve solubility significantly, further permeability studies at different pH were carried out. Prior to our studies, the effect of pH at different ethanol-aqueous concentrations on permeation was unknown and could not be accurately predicted.

### Materials and Equipment

Transport Receiver Plate (Cat #MATRNPS50) and MultiScreen-IP Filter Plate (Cat #MAIPN4550) were purchased from Millipore (Burlington, MA, USA).

Vardenafil Hydrochloride (CAS No. 224785-91-5); India Alembic Pharmaceutical Ltd, Gujarat-391450 India (Lot #1704002361).

Ethanol 190-Proof (CAS No. 64-17-5) was purchased from EMD Millipore (Burlington, MA, USA).

Acetonitrile (Cat #BDH83639.400) was purchased from BDH Chemicals (Radnor, PA, USA).

Dodecane (Cat #D221104), Sodium Phosphate monobasic (Cat #S0751), Sodium Phosphate dibasic (Cat #S0876) and Polyethylene Glycol 6000 (Cat #8.07491) were purchased from Sigma-Aldrich (St. Louis, MO, USA).

Lecithin, Refined Solid (Cat #36486) was purchased from Alfa Aesar (Haverhill, MA, USA).

Syringe Filter w/0.2 μm Cellulose Acetate Membrane (Cat #28145-475) was purchased from VWR (Radnor, PA, USA).

Polyethylene glycol 400(Lot 52081314) was purchased from EMD Millipore (Burlington, MA, USA).

### Procedure

### (a) Solution Preparation

Saturated solutions of vardenafil HCl trihydrate (5 ml) in different solvents were prepared by using an increasing amount of vardenafil and adjusted to the desired pH (range 3.5-6.0 with the use of a pH meter), as described in Example A2 above. The saturated solutions were shaken slowly at room temperature for 24 h or shaken rapidly several times and kept at room temperature for 24 h or longer. Afterwards, the solutions were filtered using a 0.2 μm filter. The filtrates were then used for permeation studies.

### (b) Permeation Studies Using PAMPA

Vardenafil permeation studies were performed using the Parallel Artificial Membrane Permeation Assay (PAMPA) with the receiver plate and multiscreen-IP filter plates. The PAMPA assay predicts passive absorption of drugs and is suitable for studies with ethanol solvents (22-23). The steady state permeation assay was carried out in triplicate or more and for a duration of 6 h or 24 h to represent steady state. For some solutions both 6 h and 24 h duration were carried out to establish consistency between the 2 durations. The donor chamber was initially coated with 5 ul of 3% (w/v) lecithin in dodecane before transferring 150 μL of desired sample into donor chamber. Then 300 μl of phosphate buffer was transferred into the acceptor wells. After 6 or 24 h of permeation, a sample was collected from the donor and receiver chamber for concentration analysis using HPLC.

Apparent permeability coefficient (Pₐₚₚ) determination, expressed as cm/sec, was calculated based on the following equation at steady state (24)

$${P}_{app} = \frac{{V}_{D} \times {V}_{A}}{( {V}_{D} + {V}_{A} ) \cdot A \cdot T} * - ln ( 1 - \frac{{C}_{A}}{{C}_{E}} )$$

where
V_{D}=volume in donor chamber
V_{A}=volume in acceptor chamber
A=effective area of the membrane (PAMPA: 0.3 cm²)
t=duration of the permeability study
C_{A}=drug concentration in the acceptor well at the end of the study
C_{E}=equilibrium concentration in both wells, and

$${C}_{E} = \frac{{C}_{A} \cdot {V}_{A} + {C}_{D} \cdot {V}_{D}}{{V}_{A} + {V}_{D}}$$

Steady state flux (Jss). The Jss, expressed as ug/sec/cm², was calculated based on the following equation

$${J}_{ss} = {p}_{app} \times {C}_{D}$$

where
C_{D}=loading concentration in the donor chamber

A comparison of Papp and Jss of Vardenafil in different solvents at pH 4 are shown in Table 4.

TABLE 4
Effect of solvents on vardenafil API permeability and flux*

| | | MeanPₐₚₚ | SDPₐₚₚ | Mean Jss | SD Jss |
|---|---|---|---|---|---|
| Row Labels | N | (cm/s) | (cm/s) | (ug/sec/cm²) | (ug/sec/cm²) |
| Water | 34 | 3.93E−09 | 5.39E−10 | 7.74E−05 | 9.17E−06 |
| Calcium | 8 | 5.34E−08 | 8.51E−09 | 8.60E−04 | 5.16E−05 |
| Lactate (3%) | | | | | |
| Calcium | 8 | 4.89E−08 | 3.17E−09 | 7.23E−04 | 4.91E−05 |
| Lactate (5%) | | | | | |
| EtOH (3%) | 6 | 4.16E−09 | 3.64E−10 | 1.17E−04 | 1.02E−05 |
| EtOH (5%) | 6 | 3.82E−09 | 4.19E−10 | 1.21E−04 | 1.32E−05 |
| EtOH (8%) | 6 | 3.68E−09 | 3.27E−10 | 1.23E−04 | 1.09E−05 |
| EtOH (12%) | 10 | 3.65E−09 | 5.63E−10 | 1.27E−04# | 1.88E−05 |
| EtOH (15%) | 6 | 3.07E−09 | 1.11E−10 | 1.16E−04 | 4.19E−06 |
| EtOH (20%) | 4 | 1.26E−09 | 6.06E−11 | 6.83E−05 | 3.28E−06 |
| EtOH (35%) | 2 | 9.81E−10 | 2.26E−11 | 9.97E−05 | 2.29E−06 |
| NMP (5%) | 6 | 2.47E−09 | 4.08E−10 | 9.52E−05 | 1.57E−05 |
| NMP (10%) | 10 | 1.63E−09 | 2.85E−10 | 7.33E−05 | 1.14E−05 |
| NMP (15%) | 6 | 1.25E−09 | 4.57E−10 | 5.85E−05 | 2.14E−05 |
| NMP (20%) | 4 | 9.51E−10 | 4.16E−11 | 5.33E−05 | 2.33E−06 |
| NMP (25%) | 30 | 3.40E−10 | 2.27E−10 | 2.70E−05 | 1.30E−05 |
| NMP (40%) | 11 | 5.85E−10 | 9.87E−11 | 7.37E−05 | 1.35E−05 |
| PEG400 (10%) | 12 | 2.09E−09 | 3.77E−10 | 5.22E−05 | 1.07E−05 |
| PEG400 (15%) | 4 | 5.35E−09 | 4.13E−10 | 1.02E−04 | 7.87E−06 |
| PEG400 (20%) | 11 | 1.80E−09 | 3.29E−10 | 4.01E−05 | 1.03E−05 |
| PEG400 (40%) | 12 | 8.12E−10 | 1.22E−10 | 1.61E−05 | 3.44E−06 |
| Calcium | 7 | 1.96E−08 | 1.31E−09 | 6.17E−04# | 9.52E−05 |
| Lactate/NMP | | | | | |
| (3%/10%) | | | | | |
| Calcium | 4 | 2.45E−08 | 9.83E−10 | 7.14E−04# | 2.86E−05 |
| Lactate/NMP | | | | | |
| (5%/10%) | | | | | |
| EtOH/Calcium | 8 | 3.32E−08 | 1.89E−09 | 7.18E−04# | 5.28E−05 |
| Lactate | | | | | |
| (12%/5%) | | | | | |
| EtOH/NMP | 9 | 1.01E−09 | 3.21E−10 | 6.06E−05 | 1.62E−05 |
| (5%/10%) | | | | | |
| EtOH/NMP | 4 | 1.02E−09 | 2.82E−10 | 7.20E−05 | 1.98E−05 |
| (12%/10%) | | | | | |
| EtOH/NMP | 8 | 4.08E−10 | 8.21E−11 | 3.29E−05 | 6.36E−06 |
| (12%/15%) | | | | | |
| EtOH/PEG400 | 6 | 4.93E−09 | 3.28E−10 | 9.21E−05 | 6.13E−06 |
| (5%/5%) | | | | | |
| EtOH/PEG400 | 10 | 3.78E−09 | 1.14E−09 | 8.53E−05 | 2.68E−05 |
| (5%/10%) | | | | | |
| EtOH/PEG400 | 8 | 4.48E−09 | 2.15E−09 | 8.03E−05 | 2.16E−05 |
| (8%/10%) | | | | | |
| EtOH/PEG400 | 6 | 4.28E−09 | 2.55E−10 | 9.03E−05 | 5.38E−06 |
| (12%/10%) | | | | | |
| EtOH/PEG400 | 10 | 6.08E−09 | 5.53E−09 | 2.03E−04# | 1.64E−04 |
| (12%/15%) | | | | | |
| EtOH/PEG400 | 6 | 3.29E−09 | 1.99E−10 | 7.64E−05 | 4.62E−06 |
| (15%/10%) | | | | | |
| EtOH/PEG400 | 6 | 3.14E−09 | 1.44E−10 | 6.44E−05 | 2.95E−06 |
| (15%/15%) | | | | | |
| PEG400/NMP | 4 | 8.59E−10 | 1.69E−10 | 3.57E−05 | 6.98E−06 |
| (15%/10%) | | | | | |

*Determined by PAMPA at room temperature and mean pH 4.0 (3.9-4.1) over 24 h; #Vardenafil organic-aqueous mixture (with saturated solubility ≥20 mg/ml) Jss much higher than Jss in water or Jss_{(ref)} (see below), p < 0.05 (−test); SD = standard deviation.

Over pH 3.5-8.0, the pH-solubility times pH-Pₐₚₚ profile of vardenafil aqueous solution becomes the pH-flux or pH-Jss profile (see profile in FIG. 10b). The pH that corresponds to its highest Jss is the pHmax with its corresponding aqueous vardenafil saturated solubility designated as V_{(ssol)pHmax}. Thus the Jss at pHₘₐₓ or Jss_{pHmax} can be used for calculating a reference Jss or Jss_{(ref)} that corresponds to the required minimum solubility, designated as Solₘᵢₙ (which is 20 mg/ml for a minimum IN dose of 2 mg/nostril per previous calculation in [0061]):

$${Jss}_{( ref )} = {Jss}_{pHmax} ( {Sol}_{min} / {V}_{( ssol ) pHmax} )$$

where Solₘᵢₙ is the required minimum solubility (which is 20 mg/ml for a minimum IN dose of 2 mg/nostril vardenafil, or a higher value corresponding to a higher IN dose); V_{(ssol)pHmax)} is the aqueous vardenafil saturated solubility at pHₘₐₓ (i.e. pH4 per FIG. 10b) which is 18.19 (from Table 3). Using Solₘᵢₙ=20, V_{(ssol)pHmax)}=18.19, Jss_{pHmax}=7.74 E-05 ug/s/cm² (from Table 4), Jss_{(ref)}=8.5E-05 ug/s/cm² for a required vardenafil IN dose of 2 mg/nostril.

Any vardenafil organic-aqueous mixture at any pH (within the range of pH3.5-7.5) with solubility of ≥20 mg/ml that has a corresponding Jss value ≥Jss_{(ref)} can qualify for IN vardenafil formulation.

### (c) HPLC Analysis

After mixing 100 μL of donor or receiving chamber sample with 100 μL of MeOH and 10 μL of internal standard for 2 minutes, the sample was centrifuged at 10,000 rpm for 10 mins before HPLC injection. The loading stock solutions were diluted with 50% MeOH before HPLC injection. The HPLC assay was performed as described in Example A2.

### Results

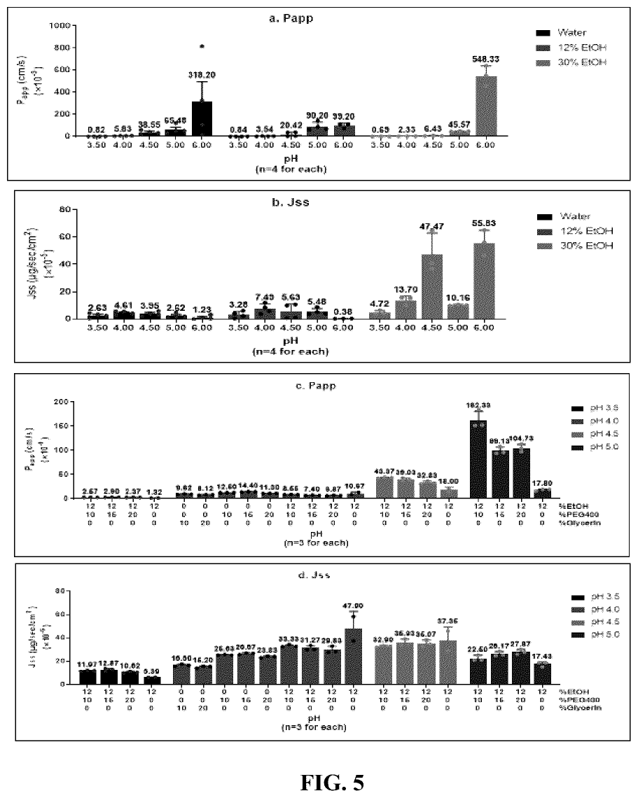

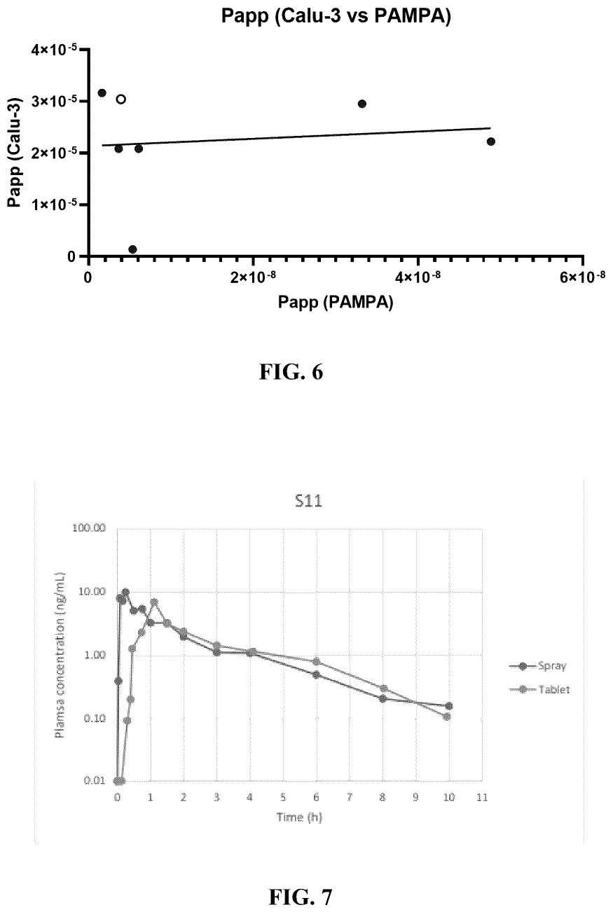

The results of the comparative permeation study shown in Table 4. An excellent correlation of Papp results between these 6 h and 24 h durations of permeation study were observed, showing consistency of the Papp experiment. Additional individual studies using, e.g., pure aqueous solution and other ethanol-aqueous mixtures also supported a similar trend of pH influence on permeability, as shown in FIG. 5.

### Conclusion

As disclosed herein, vardenafil API has increasing solubility as pH decreases. However, vardenafil permeability (Papp) increases with increasing pH (corresponding to higher % of unionized species) is theoretically expected as pH increases from 3.5 to 5.0 (FIG. 5. Drug flux or Jss (a parameter composed of Pₐₚₚ times saturated solubility) appears optimal at pH 4.0 for an vardenafil aqueous solution (FIG. 5b). The representative vardenafil organic-aqueous(EtOH(12%)-aqueous) solution also shows a similar trend (FIG. 5B). Based on these data, JSSₘₐₓ is selected to be at pH4.

A number of organic-solvents with a solubility of ≥20 mg/ml at pH4, have been discovered to also significantly exceed the mean vardenafil Jss form the aqueous solution or Jss (ref) (8.5E-05 ug/s/cm²), and can be considered as suitable formulation for IN vardenafil (Table 4).

Many organic-aqueous mixtures or organic-organic-aqueous mixtures, can improve solubility and permeability of vardenafil HCl trihydrate, the current active pharmaceutical ingredient for vardenafil. On the hand a combination of two different organic solvents in water may improve permeability even though the combination may not improve solubility compared to the single organic-aqueous mixture, such as 12% EtOH-aqueous, for example. Without being limited or constrained by any theory, it is believed that the solvents described herein and other suitable organic-aqueous solvents that can improve solubility of different phosphodiesterase inhibitors at the pH range close to the physiologic pH at the nasal or sublingual sites can also improve their permeation and be suitable for nasal and sublingual delivery.

### Example B2

### Determination of Vardenafil Permeability in Selected Solutions Using a Calu-3 Cell Line Model

This example describes the determination of permeability of vardenafil using a Calu-3 cell line model.

As disclosed herein, the permeability of vardenafil API in different solvents was screened using the in vivo cell line model Calu-3 (a non-small-cell lung cancer line). Although screening the effect of different solvents on vardenafil permeability can be efficiently carried out using the PAMPA method, the reliability of this method can be improved if confirmed using a physiological model, such as a cell line resembling the target membrane. Thus for the present study, Calu-3 cell line which is a suitable model for IN permeation is used to confirm the permeability of the PAMPA study results.

The Pₐₚₚ of aqueous soluble drugs determined by the Calu-3 cell line model has been shown to be related to the IN absorption in animal studies when determined at pH 7.4 (23-24). As disclosed herein, the Calu-3 cell line model was utilized for confirmation of the relative values of Pₐₚₚ of various organic-aqueous solutions in comparison to that in water at pH 4.0 (to simulate IN administration of vardenafil formulation).

### Materials

Glacial acetic acid (>99% pure, CAS 64-19-7) was purchased from Alfa Aesar (Haverhill MA, USA).

Acetonitrile ≥99.5% ACS (CAS No. 75-05-8) was purchased from VWR Chemicals BDH®.

Sodium Phosphate Monobasic Monohydrate (Phosphate Buffer) was purchased from BDH Chemicals (Radnor, PA, USA).

NaOH (Sodium Hydroxide) was purchased from Biobasic Canada Inc. (Markham, Ontario, Canada).

Sodium Phosphate Dibasic, Heptahydrate (Phosphate Buffer) was purchased from EMD Millipore (Burlington, MA, USA).

Vardenafil Hydrochloride Trihydrate USP was purchased from SMS pharmaceuticals Ltd. (India).

Gibco Hank's Balanced Salt Solution (HBSS), o-Phosphoric Acid, 85% (HPLC), Transwell™ Multiple Well Plate with Permeable Polycarbonate Membrane Inserts Triethylamine was purchased from Thermo Fisher Scientific (Waltham, MA, USA).

Syringe Filter w/0.2 um Cellulose Acetate Membrane was purchased from VWR part of Avantor (Radnor, PA, USA).

### Equipment

Accumet Basic pH meter was purchased from Fisher Scientific (Leicestershire, UK).

Agilent 1260 Infinity HPLC system which consisted of a G1311B 1269 Quat Pump, a G7129 1260 vial sampler, and a G1315D 1260 DAD VAL detector was purchased from Agilent (Santa Clara, CA).

Analytical Balance was purchased from Mettler-Toledo, LLC (Columbus, OH).

Water used was obtained from Nanopure Water Filtration System (Barnstead Nanopure Diamond Life Sci UV/UF system (Cat #D119310 purchased form APS Water Servives Corporation (Lake Balboa, CA, USA).

Transepithelial electrical resistance (TEER, in Ohm·cm2) determination equipment.

### Procedure

### (a) Solution Preparation

Saturated solutions of vardenafil (2 mg/mL) in different solvents were prepared and adjusted to the desired pH (range 3.9-4.1 with the use of a pH meter), as described in Examples A2 and B1 above. Various solutions containing vardenafil at 2 mg/ml were prepared by fully dissolving each powder by vortexing, followed by overnight mixing on a rotating platform. Afterwards, the solutions were filtered using a 0.2 μm filter. The filtrates were then used for permeation/permeability studies.

### (b) Culture of Calu-3 and Preparation of Monolayers

The study was carried out similar to that described previously (26-27). Calu-3, a human bronchial submucosal gland carcinoma cell line, was grown in DMEM:Ham's F-12 (1:1) mixture supplemented with 10% FBS and 1% penicillin/streptomycin solution. The cells were harvested with 0.25% trypsin-EDTA and seeded on polycarbonate filters (pore size: 0.4 μm, growth area: 1.12 cm², 12 wells/plate, Corning) at a density of 5×10⁵ cells/well. The culture medium was changed every 2 days over the course of the experiment. The monolayer was used for in vitro transport studies, 9-10 days after seeding.

### (c) In-Vitro Permeation/Permeability Study Using Calu-3 Cell Line Model

After the TEER of the monolayer was measured and found around 500 Ohm·cm², the growth medium was aspirated and the upper and lower chambers washed with a transport medium (TM: Hank's balanced salts solution supplemented with 15 mM glucose and 10 mM HEPES buffer, pH 7.4).

Following 10 min of incubation, the solution in the apical chamber was replaced the studied vardenafil solutions at pH 4.0. Aliquots of the sample (50 μL) were taken from the basal side at 15, 30, 45, 60, 90, 120 mins under a BSL2 hood. A 50 μL aliquot of transport medium was added to replenish the volume each time, and TEER measurements were determined periodically.

### (d) HPLC Assay Preparation and Measurement

50 μl samples from the receiving and apical chambers were either mixed with 50 μl of 50% MeOH and 10 μl internal standard or diluted with 50 μl medium and internal standard. Supernatant was taken after centrifugation for HPLC analysis. HPLC Analysis was performed as outlined in Examples A2 and B1.

### (e) Calculation of Papp

Pₐₚₚ, expressed in cm/sec, was calculated from the samples analyzed by HPLC using the equation:

Pₐₚₚ=dQdt/(AC); wherein

dQdt is the appearance of drug in the receiving chamber (nmol/sec), A is the surface area of the monolayer (1.12 cm²), and C is the initial concentration of drug in the apical chamber.

### Results

The calculated, mean Pₐₚₚ of different vardenafil solutions are listed in Table 5. The majority of Pₐₚₚ values were close to the Pₐₚₚ value of vardenafil in water, except for the Pₐₚₚ value of vardenafil in 15% PEG400 solution. For vardenafil in 15% PEG400 solution, the TEER values increased to about 1200 Ohm·cm² above initial baseline value and gradually drop to a level similar to the baseline value (about 500 Ohm·cm²) at 120 min. Adding EtOH/PEG(12%/15%) also increased the initial TEER about 1200 Ohm·cm², but returned more quickly to a level similar to baseline at 40 min. Most other solutions usually resulted in a rapid decrease of TEER to below 500 Ohm·cm² baseline value in less than 20-40 min.

TABLE 5
Vardenafil API permeability using a Calu-3 cell line model*

| | Number | Mean Pₐₚₚ (cm/s) | SD of Pₐₚₚ (cm/s) |
|---|---|---|---|
| Water | 3 | 3.04E−05 | 4.17E−06 |
| Acetic Acid (2%) | 2 | 3.57E−05 | 1.62E−06 |
| Calcium Lactate (5%) | 2 | 2.22E−05 | 2.77E−06 |
| EtOH (12%) | 2 | 2.08E−05 | 5.73E−06 |
| NMP (10%) | 2 | 3.16E−05 | 2.69E−06 |
| PEG400 (15%) | 2 | 1.36E−06* | 6.10E−07 |
| Acetic Acid/NMP (2%/10%) | 3 | 4.99E−05 | 1.43E−05 |
| EtOH/Calcium Lactate (12%/5%) | 3 | 2.95E−05 | 1.06E−05 |
| EtOH/NMP (12%/10%) | 3 | 2.60E−05 | 3.86E−06 |
| EtOH/PEG400 (12%/15%) | 3 | 2.08E−05 | 4.86E−06 |
| HBSS (pH 4) | 1 | 3.60E−05 | n/a |

*Determined in 37° C., at pH 4.0 (3.9-4.1); Pₐₚₚ = apparent permeability; SD = standard deviation; duration of permeation was ≤2 h.
*P < 0.01 (t-test) compared to water

### Conclusion

In this experiment, the relative Papp values of the solutions also directly reflect relative Jss values since the concentration of each solution is 2 mg/ml. The Pₐₚₚ values of vardenafil API in different organic-aqueous solutions are approximately the same as the Pₐₚₚ value of vardenafil in water for most of the solutions, except 15% PEG400 solution, which is 20-fold lower (p<0.001, 2 sample t-test). This may correspond with the increased TEER (above that of the initial TEER of the medium, or 500 Ohm·cm²) of the organic-aqueous solution containing vardenafil with 15% PEG400 solution. The increase in TEER may indicate an increasing tight junction function of the cell membrane. In support of this hypothesis, the addition of ethanol to PEG400—ethanol being a known to enhance membrane permeability—had significantly lower TEER values. Thus, the use of Calu-3 model to further screen vardenafil formulations initially identified by PAMPA can be a useful step in eliminating undesirable formulations.

### Example C1

### Methods of Administering Phosphodiesterase Inhibitors

This example describes methods of intranasal and sublingual administration of phosphodiesterase inhibitors.

A phosphodiesterase inhibitor is added to a mixed organic-aqueous solvent, and the pH of the solvent is adjusted. Any phosphodiesterase inhibitor can be used, including vardenafil (Levitra), sildenafil (Viagra), and tadalafil (Cialis), for example. Addition of the phosphodiesterase inhibitor to an organic-aqueous solvent at a given pH can result in increased solubility and or permeability of the phosphodiesterase inhibitor. Any organic-aqueous mixture or solvent can be used, including any organic-aqueous solvent that is relatively safe or well tolerated by a human subject and that is capable of sufficiently solubilizing and enhancing permeation of the phosphodiesterase inhibitor.

Improved solubility of the phosphodiesterase inhibitor can result in improved flux (solubility times permeability or Jss) of the phosphodiesterase inhibitor, such as vardenafil, sildenafil, or tadalafil, for example, across a mucosal membrane. Mucosal flux of phosphodiesterase inhibitors in an organic-aqueous mixture is suitable at a pH range of about 4.0 to about 8.0. As a result of improved flux across the mucosal membrane, Cmax and bioavailability of the phosphodiesterase inhibitor can be increased and Tmax can be shortened in comparison to oral administration of the same phosphodiesterase inhibitor. This will translate to higher plasma concentration and faster onset of action upon intranasal or sublingual administration as compared to oral administration of the same dosage of the phosphodiesterase inhibitor.

A phosphodiesterase inhibitor can be delivered via nasal or sublingual membrane in any dosage form, but a solution formulation, e.g. using a spray should result in the fastest onset of effect compared to other dosage forms. As an example, an amount of phosphodiesterase inhibitor can be added to a mixed organic-aqueous solvent to deliver a desired amount of the phosphodiesterase inhibitor in a 100 μl volume per spray, either intranasally or sublingually.

The phosphodiesterase inhibitors with improved solubility and permeation as described above are administered for the treatment of erectile dysfunction, for example.

In addition to phosphodiesterase inhibitors, this method can be applied to any ionizable compounds.

### Example C2

### Comparison of IN Vs Oral Administration of Vardenafil in Rats

This example describes the determination of bioavailability of vardenafil following either oral or IN administration, as well as how variations to the formulation affects the pharmokinetics.

IN administration allows compounds to bypass liver metabolism. In addition due to thin layer of mucosal nasal membrane, IN administration allows for quicker absorption, greater bioavailability, and faster peak concentration time (Tmax) for drugs compared to oral administration, if an appropriate drug formation is administered. Certain formulations have been found to have different vardenafil Jss or Pₐₚₚ in vitro. Thus, these differences in formulation are likely to influence the above pharmacokinetic parameters when administered via the IN route. Prior to our studies, the stepwise approach of estimating an appropriate dose/drug concentration, and identifying and confirming specific formulations with desired solubility and permeability for IN administration to achieve an improved and appropriate vardenafil plasma concentration relative to oral administration of the dose were unknown.

### Procedure

Vardenafil aqueous solution and organic-aqueous solutions were prepared using the same protocol as described in Example A2. Sprague Dawley rats with jugular vein cannula inserted were ordered from Envigo RMS, Inc. (Indianapolis, IN). After arrival, the rats were adjusted to the animal vivarium environment of the Western University of Health Sciences for one week before the pharmacokinetic study. The anesthesia and study procedure were carried out similar to a previously published study (29). A total of 6 formulations were tested for IN administration and one for PO dosing via gavage (see Table 6 below).

A subset of rats were given formulations per os (PO, or orally) containing vardenafil (5.6 mg/kg). The other subset of rats were given formulations intranasally (IN) containing vardenafil (2.8 mg/kg). The IN rat group was given formulations through a micropipette at about 12.5 ul administration per nostril. Rats given formulation of water and PEG formulation by IN route were 1.7 mg/kg and 2.0 mg/kg respectively. The rats were randomly assigned to receive 6 formulations (see Table 6).

After administration of the formulations, 200 uL of blood were obtained from rats at 0, 2, 5, 10, 15, 20, 30, 45, 60, 120, 180 minutes. One week after treatment, the hematocrit levels of the rats returned to normal, as verified by the blood plasma of randomly selected rats in the study. Based on such hematocrit response, general physical activity, and patency of the cannula, the rats were crossed over to a different formulation treatment one week later with same blood samples collected. After completion of any blood sampling, the samples were centrifuged and plasma collected and stored for analysis using Liquid Chromatography Tandem Mass Spectrometry (LC/MS/MS, Sciex API4000 and Agilent HPLC 1200 system) The assay used rat plasma to construct the standard curve and sildenafil was used as the internal standard (IS). The assay procedure was similar to that of the human study as described below (see Determination of the plasma concentration of vardenafil under EXAMPLE C3).

### Results

The standard curve for the assay of the vardenafil concentration determination in rat plasma showed excellent correlation coefficient (R²=0.9981 for the concentration of 0.1-1000 ng/ml). The vardenafil formulation and number of rats received the IN and PO dosages are shown in Table 6. The pharmacokinetic parameters of oral vs IN administered formulation of ethanol/PEG400(12%/15%) are shown in Table 7. The Pharmacokinetics of IN administered formulation 2 to 6 are shown in Table 8.

TABLE 6
Formulations containing vardenafil administered
to rats either PO or IN

| # | Formulation | N | Route | dose | |
|---|---|---|---|---|---|
| 1 | EtOH/PEG400 (12%/15% | 3 | PO | 5.6 | mg/kg |
| 2 | EtOH/PEG400 (12%/15% | 3 | IN | 2.8 | mg/kg |
| 3 | EtOH 12% | 3 | IN | 2.8 | mg · k |
| 4 | PEG40015% | 2 | IN | 2.0 | mg/kg |
| 5 | PEG400/NMP(15%/10%) | 4 | IN | 2.8 | mg/kg |
| 6 | Water | 3 | IN | 1.7 | mg/kg |

TABLE 7
Pharmacokinetic parameters of vardenafil following nasal
and oral administration of ethanol/PEG400(12%/15%)

| | | Nasal | Oral |
|---|---|---|---|
| Parameters | Units | Mean ± SD | Mean ± SD |
| T_{1/2} | min | 59.19 ± 13.66 | 137.42 ± 78.57 |
| Tₘₐₓ | min | 6.67 ± 2.88 | 16.67 ± 12.58 |
| *Cₘₐₓ | ng/ml | 30.79 ± 16.87 | 12.18 ± 7.22 |
| *AUC_{0-inf} | ng/mL*min | 1622.57 ± 712.45 | 982.10 ± 300.94 |

*Normalized per mg/kg dose

TABLE 8
Pharmacokinetic parameters of vardenafil following IN administration of 5 formulations*

| Parameters | Units | F2 | F3 | F4 | F5 | F6 |
|---|---|---|---|---|---|---|
| T_{1/2} | min | 59 ± 13 | 46 ± 45 | 55 ± 20 | 48 ± 79 | 54 ± 201 |
| Tₘₐₓ | min | 7 ± 3 | 12 ± 3 | 16 ± 20 | 6 ± 3 | 4 ± 2 |
| Cₘₐₓ | ng/mL | 31 ± 17 | 40 ± 13 | 13 ± 2 | 10 ± 3 | 33 ± 14 |
| AUC_{0-inf} | ng/mL · min | 1622 ± 712 | 2262 ± 567 | 812 ± 42 | 508 ± 187** | 1728 ± 656 |

F = formulation,
*Normalized per mg/kg dose.
**P < 0.01 (t-test) compared to F6 (water formulation)

### Discussion and Conclusion

The results of the relative bioavailability (AUC), Cmax (peak concentration) and Tmax (time of Cmax) are consistent with that expected from formulations identified in Examples A2, B1, B2 and C1. Solvents with low Papp/flux relative to that in water e.g. vardenafil in 15% PEG400 or in 15% PEG-10% NMP from in vitro PAMPA and/or Calu-3 studies can lead to relatively low bioavailability when administered by IN route. Compared to water formulation (F6), the AUC of F5 is significantly lower. The AUC and Cmax of formulation #4 are also substantially lower compared to that of formulation F6, however the values are not statistically significant at p<0.05 due to small number of rats completing the study. However, solvents with similar/better Papp/flux as that in water, e.g. vardenafil in 12% ethanol and in ethanol/PEG400(12%/15%) can lead to similar/better bioavailability as that in water. Thus, these rat data confirm the present method of screening and selection for desired formulation using the stepwise approach demonstrated in Examples A1, A2, B1, and B2.

### Example C3

### Comparison of IN Vs Oral Administration of Select Phosphodiesterase Inhibitors in Humans

This example describes the determination of bioavailability of vardenafil following either oral or IN administration in humans, as well as how variations to the formulation affects the pharmakinetics.

As disclosed herein, this study compared an SDS-089 Solution (composed of vardenafil 20 mg/ml dissolved in 12% EtOH/15% PEG400) administered as Nasal Spray (IN administration) to the Levitra Oral Tablet 10 mg (oral, or PO administration). The steps for selecting >20 mg/ml vardenafil solubility, its solution in 12%ethanol-15% PEG400 for nasal drug delivery are described in Examples A1, A2, B1, B2 and C1.

### Materials

Vardenafil nasal spray solution (SDS-089 nasal spray) was prepared for each human subject participating in the study as prescribed by the Principal Investigator.

The active pharmaceutical ingredient is from Alembic Pharmaceutical Ltd., India (batch 1704002361) which meets USP standard.

The nasal spray solution was composed of vardenafil API 20 mg/ml solubilized in 12% ethanol and 15% PEG400 at pH about 4.0. The SDS-089 nasal spray solution was filtered (0.22 μm filter) and transferred to a small volume 5 mL amber bottle, fitted with nasal spray device to deliver 100 μL per spray (manufactured by Aptar, Pharma, France). The ability of the Aptar nasal spray device to deliver 100 uL per spray was verified prior to the pilot clinical study. The spray delivered 2 mg vardenafil HCl dissolved in 12% ethanol and 15% PEG400 solution per spray.

Vardenafil HCl (Levitra) 10 mg oral tablet which was manufactured by Bayer Pharmaceutical (NDC: D173-0830-13, Lot #: 5930248) was purchased from a pharmacy.

SDS089 was prepared by a licensed pharmacy technician under supervision of a licensed pharmacist at the Medical Center in the Patient Care Center of Western University of Health Sciences, Pomona, California, USA.

### Procedure

The twelve human subjects recruited for the study were healthy volunteers between 21 and 45 years old. Each subject received two study treatments: SDS-089 Solution as Nasal Spray (4 mg vardenafil HCl trihydrate) and Levitra Oral Tablet (10 mg) in a randomized sequence, separated by a period of 7±1 days.

On the day of study, the subjects had an intravenous catheter inserted. All subjects were given either the 10 mg Levitra tablet orally PO) or the 4 mg SDS-089 solution (2 mg/spray per nostril) Nasal Spray through IN administration. Administration was followed with 240 ml water. After a period of 1 week, the treatment was crossed over in each subject (such that those subjects who previously were given IN administration were now given PO administration, and subjects who previously were given PO administration were now given IN administration). Each subject took 240 ml of water with drug administration and was allowed to drink water and clear liquids 2 hours post single-dose treatment. Meals were provided and given at least 4 hours post dose.

A total of 17 blood samples (2 cc each) were collected following administration of the drug formulations. The blood samples were collected at 0 (pre-dose), 2 min, 5 min, 10 min, 15 min, 30 min, 45 min, 60 min, 90 min, 2 h, 3 h, 4 h, 6 h, 8 h, and 10 h. All blood samples were immediately centrifuged at 3000 rpm for 10 minutes and stored at −80° C. until ready for bio-analysis.

During the study, the safety assessments included adverse event monitoring, vital signs, and targeted history and physical examinations as needed as per the judgment of the medical supervisor.

### (a) Determination of the Plasma Concentration of Vardenafil

Analysis of the vardenafil concentration in blood plasma was conducted using Liquid Chromatography Tandem Mass Spectrometry (LC/MS/MS, Sciex API4000 and Agilent HPLC 1200 system), via a contract service provided by Dr. Stan Louie of the University of Southern California, School of Pharmacy, analytical certified laboratory. During the validation experiments, each calibration standard and QC sample was prepared by spiking of a specified amount of vardenafil HCl (from USP) and Sildenafil (from LETCO) served as the internal standard into blank human plasma. A 50 ul aliquot of analytes was extracted using 850 ul methanol, centrifuged, and the top supernatant dried. The powder was then reconstituted with 60 ul of 50% methanol and 30 ul injected to LCMS after filtration. The isolated analytes were separated using reverse-phase high performance Eclipse plus C18 column (Agilent) with the following dimensions, 4.6×100 mm, 3.5 μm particle size. The concentration of analytes in each standard was quantified using a triple quadrupole tandem mass spectrometer operating in positive mode with electrospray ionization mode (ESI). Vardenafil and sildenafil were detected using multiple-reaction-monitoring (MRM) for each of the respective analyte. The average assay accuracy ranged from 92-110%. The R² of the calibration curves ranged 0.9977 to 0.9998. Precision, defined as the coefficient of variation (CV)=(standard deviation/mean of replicate measurements×100%) ranged from 4-8%. The lower limit of quantitation was 0.05 ng/ml.

### Results

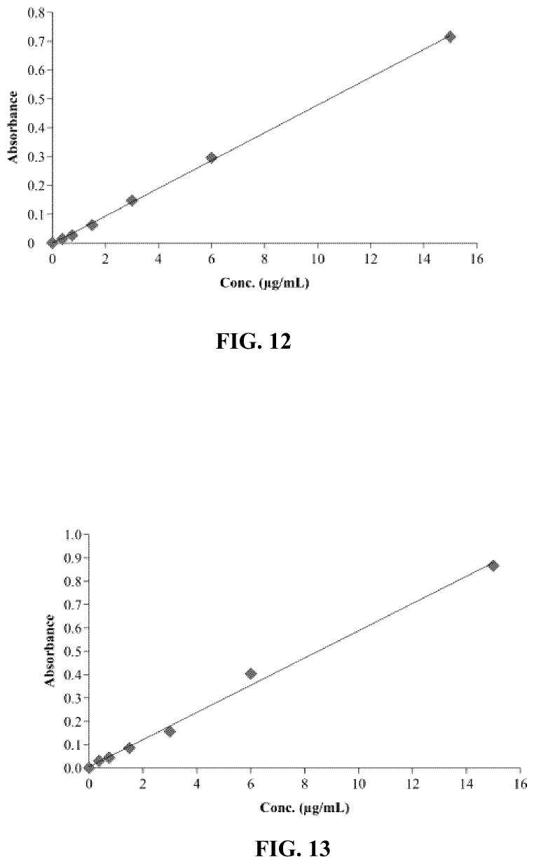

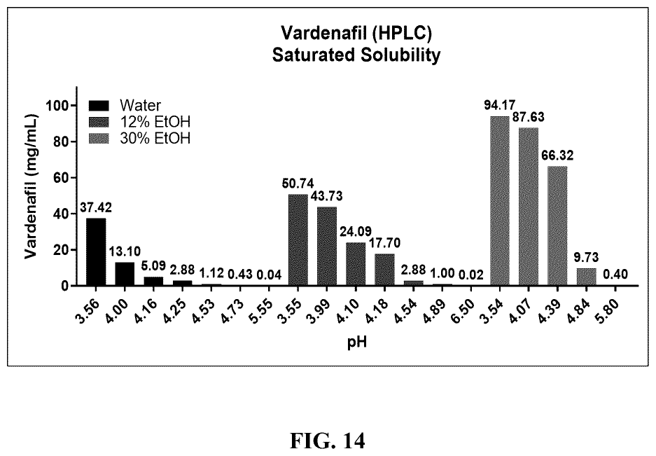

A representative vardenafil concentration time curve is shown in FIG. 12 and mean comparative pharmacokinetic parameters are shown in Table 9. Based on the area under the concentration-time-curve from 0 to infinity (AUC_{0-inf}), the overall bioavailability of SDS nasal spray was calculated to be about 1.4 times that of the oral vardenafil. The time of maximum concentration (Tₘₐₓ) occurred at a median time of 10 min following SDS089 nasal spray compared to 58 min following the oral table. (The Tₘₐₓ was 6-15 min in 85% of subjects receiving IN dosing compared to 45-60 min in 92% of subjects receiving oral dosing) The maximum concentration (Cₘₐₓ) following SDS089 was within the range of Cₘₐₓ following the oral dose. These data showed that the bioavailability of SDS089 nasal spray 4 mg is close to that of 10 mg oral dose, but with a much shorter Tₘₐₓ.

TABLE 9
Comparison of mean pharmacokinetic parameters of nasal spray vs oral tablet
Non-compartmental Model

| | | Nasal spray (4 mg) | | Oral tablet (10 mg) | |
|---|---|---|---|---|---|
| Parameters | Units | Average | Range | Average | Range |
| λ [symbol] | 1/h | 0.305 ± 0.085 | 0.148-0.416 | 0.304 ± 0.084 | 0.183-0.423 |
| T_{1/2} | h | 2.517 ± 0.987 | 1.665-4.688 | 2.463 ± 0.737 | 1.640-3.778 |
| Tₘₐₓ | h | 0.162 ± 0.544 | 0.083-2.000 | 0.971 ± 0.349 | 0.750-2.000 |
| Cₘₐₓ | ng/mL | 9.216 ± 4.109 | 3.550-16.500 | 11.236 ± 7.830 | 2.040-27.900 |
| AUC_{0-inf} | ng/mL*h | 21.025 ± 15.538 | 10.010-67.069 | 37.252 ± 41.514 | 7.436-156.896 |

Note:
The average values of above pharmacokinetic parameters are the mean value, except for Tₘₐₓ values are the median value to represent a more meaningful representation of Tₘₐₓ. [symbol]
indicates data missing or illegible when filed

Over the two treatment periods, 47 adverse events (AEs) occurred during the study. Of the 47 AEs, 42 were recorded to be an adverse drug reaction (ADR), in which 33 ADRs were associated with the nasal spray and 9 ADRs were associated with the oral tablet. Adverse effects observed included headache, sneezing, running nose, watery eyes, nasal irritation, and throat irritation. Although the SDS-089 nasal spray formulation caused more nasal symptoms to occur, overall the adverse reactions were transient and well-tolerated by the subjects. The reported headaches showed no relation when mild and moderate headaches were correlated with Cₘₐₓ and AUC_{0-inf}.

### Conclusion

As disclosed herein, the study compared nasal to oral administration of vadenafil in twelve healthy volunteers. Adverse effects were more common in IN administration, however, these effects appeared transient and tolerable. The overall results of this study were consistent with those in rats, wherein the IN administration achieved an earlier Tmax and better bioavailability. These human study results further confirm that an appropriate formulation and dose can be identified using the stepwise approach as described in Examples A1, A2, B1, and B2.

### REFERENCES

Each of the following references is incorporated by reference in its entirety herein.
1. Prescribing information for sildenafil (Viagra), 2017.
2. Prescribing information for tadalafil (Cialis), 2018
3. Kim J. S., Kim M S, Baek I H. Enhanced Bioavailability of Tadalafil after Intranasal Administration in Beagle Dogs. Pharmaceutics. 2018 Oct. 15; 10(4). pii: E187. doi: 10.3390/pharmaceutics 10040187.
4. Prescribing information for vardenafil (Levitra), 2014.
5. Bhise, S. B, Yadav A V, Avachat A M, Malayandi R., Bioavailability of intranasal drug delivery system. Asian J Pharmaceutics 2008; 2:201-215.
6. Romeo, V D, deMeireles J, Sileno A P, Pimplaskar H K, Behl C R. Effects of physicochemical properties and other factors on systemic nasal drug delivery. Advanced Drug Delivery Reviews. 1998; 29:89-116.
7. Wang, Y., Chow, M. S. S, Zuo, Z., Mechanistic analysis of pH-dependent solubility and trans-membrane permeability of amphoteric compounds: application to sildenafil. Int. J. Pharm., 2008; 352, 217-224.
8. Chhajed, S, Sangale S, Barhate S D. Advantageous nasal drug delivery system: a review. http://ijpsr.com/bft-article/advantageous-nasal-drug-delivery-system-a-review/?view=fulltext; DOI: http://dx.doi.org/10.13040/IJPSR.0975-8232.2(6).1322-36.
9. Aurora, J. Development of nasal delivery systems. A review. Drug Deliv Technol 2002; 2:1-8.
10. Beule A. G. Physiology and pathophysiology of respiratory mucosa of the nasal and the paranasal sinuses. GMS Curr Top Otorhinolaryngol Head Neck Surg. 2010; 9: 1-24.
11. Bitter, C., Suter-Zimmermann, K., & Surber, C. (2011). Nasal drug delivery in humans. Curr Probl Dermatol, 40, 20-35. https://doi.org/10.1159/000321044.
12. Park J, Agyemang A, Chow M. S. S. (2019).Can current available drugs for erectile dysfunction be re=formulated to achieve rapid effect? J. Asian Assoc of Schools of Pharmacy 8, 58-63.
13. ScienceFinder. https://www.scifinder.com.
14. Malpani, A., Pankaj, W., Belonkar, N. V., (2009), Aqueous solubility measurement and prediction tools. Pharmainfolnet Latest Reviews, 7, 1-15.
15. Eric, S., Kalinic, M., Popovic, A. et al, (2020), the importance of the accuracy of the experimental data for the prediction of solubility, J. Serb. Chem. Soc., 75, 483-495.
16. Berben P, Bauer-Brandl A, Brandl M, Faller B, Flaten G E, Jacobsen A C, Brouwers J, Augustijns P. Drug permeability profiling using cell-free permeation tools: Overview and applications. Eur J Pharm Sci. 2018 Jul. 1; 119:219-233. doi: 10.1016/j.ejps.2018.04.016. Epub 2018 Apr. 13.
17. Kansy, M., Senner, F., Gubernator, K., (1998), Physicochemical high throughput screning:Parallel artificial membrane permeation assay in the description of passive absorption processes. J. Med. Chem., 41, 10071010
18. Wohnsland, F., Faller, B. High-throughput Permeability pH Profile and High-throughput Alkane/Water Log P With Artificial Membranes, J. Med. Chem., 2001; 44, p. 923-930.
19. Nielsen P E, Avdeef A. (2004). PAMPA-a drug absorption in vitro model 8. Apparent filter porosituy and the unstirred water layer. European J Pharmaceutical Sciences 22: 33-41
20. Birch, H., Redman, A. D., Letinski, D. J., Lyon, D. Y., & Mayer, P. (2019). Determining the water solubility of difficult-to-test substances: A tutorial review. Anal Chim Acta, 1086, 16-28. https://doi.org/10.1016/j.aca.2019.07.034
21. He, Y., Ho, C., Yang, D., Chen, J., & Orton, E. (2017). Measurement and Accurate Interpretation of the Solubility of Pharmaceutical Salts. J Pharm Sci, 106(5), 1190-1196. https://doi.org/10.1016/j.xphs.2017.01.023
22. Bergstrom, C., Avdeef, A., (2019), Perspectives in solubility measurement and interpretation, Admet & DMPK 7, 88-105
23. Inoue, D., Furubayashi, T., Tanaka, A. et al (2020), Quantitative estimation of drug permeation through nasal mucosa using in vitro membrane permeability across Calu-3 cell layers for predicting in vivo bioavailability after intranasal administration to rats, European J. Pharmaceutics and Biopharm., 149,145-153
24. Furubayashi, T., Inoue, D., Nishiyama, N., et al (2020), Comparison of various cell lines and three-dimensional mucociliary tissue model systems to estimate drug permeability using an in vitro transport study to predict nasal drug absorption in rats, Pharmaceutics 12, 1-14
25. Wang, Y, Zuo, Z, Chow, MS (2008). HQ-lu-1 model for screening sublingual drug delivery—influence of pH, osmolarity and permeation enhancer. Int J Pharm. 209 March 31; 370(1-2):68-74. Doi: 10.1016/j.ijpharm.2008.11.010
26. Berry B, Altman P, Rowe J, Vaisman J. Comparison of Pharmacokinetics of Vardenafil Administered Using an Ultrasonic Nebulizer for Inhalation vs a Single 10-mg Oral Tablet. J Sex Med. 2016 July; 13(7):1111-8. doi: 10.1111/j.1743-6109.2009.01403.x. PMID: 27318021.
27. https://www.accessdata.fda.gov/drugsatfda_docs/nda/2012/202813Orig1s000SumR.pdf
28. http://www.druginformation.com/RxDrugs/B/Beclomethasone %20Dipropionate %20HFA.html
29. Qian, S, Wong, Y C, Zuo, Z. (2014), Development, characterization and application of in situ gel systems for intranasal delivery of tacrine. Int J Pharm. 2014 Jul. 1; 468(1-2):272-82. doi: 10.1016/j.ijpharm.2014.04.015. Epub 2014 Apr. 5. PMID: 24709220.
30. Lu, H T., Chen, R. Sheu, M et al (2011), Rapid-onset sildenafil nasal spray carried by microemulsion systems: in vitro evaluation and in vivo pharmacokinetic studies in rabbits. Xenobiotica DOI: 10.3109/00498254.2011.563877

### Example D1

### Intranasal (IN) Dosing and Formulation of Sildenafil

Properties of sildenafil including its solubility and stability was determined in various organic-aqueous mixtures.

Sildenafil is approved as oral tablet by FDA for treatment of ED with the usual dosage of 25 to 100 mg (1). Its active pharmaceutical ingredient, sildenafil citrate, has a molecular weight of 666.7 g/mole the sildenafil base has a molecular weight of 474.6 g/mole (1). The sildenafil base has a pKa=7.2 and log P=2.7 (12). Its solubility is about 7.0 mg/ml in water at pH 3-4, 2.1 mg/ml pH 5 and 0.11at pH 6, 0.03 mg/ml at pH 7 (7). Such solubility in water is too low to develop a suitable IN formulation. (Assuming bioavailability from IN administration is 2× better than that from the oral administration, to achieve effective concentration equivalent to at least 25 mg oral dose will require a solubility of sildenafil base of about 42 mg/ml. Such concentration is calculated based on desirable spray volume of 0.150 ml per nostril so that the concentration needed is 12.5 mg/0.3 ml=42 mg/ml for both nostrils). Thus, despite an excellent membrane partition coefficient (log P=2.7), intranasal administration using aqueous sildenafil is not possible to achieve an effective IN dose (equivalent to at least 25 mg oral dose) even at the most soluble pH range (pH 3-4).

Sildenafil citrate, however, can achieve an improved solubility in certain oil (e.g. oleic acid, safflower oil) or surfactants (e.g. Tween 20), cremophor RH60, cremophor EL) or co-surfactants (e.g. PEG200) (30). A microemulsion system consisting of 40% oleic acid, 10% H2O, and 50% Tween 80 ethanol (at a 1:4 weight ratio) for nasal sildenafil preparation was found to produce rapid action (30). However, such oily solution is unlikely to be suitable for normal use via nasal spray. Thus a new formulation with no oil content consisting of simple organic-aqueous mixture which is safe but also can achieve suitable solubility and permeability will be most desirable.

### Example D2

### Screening for Solubility and Stability Characteristics of Various Organic-Aqueous Mixtures: Sildenafil

As an example, using the method described for vardenafil IN spray formulation, the solubility of saturated sildenafil in water at different pH was first screened, followed by screening the permeability at different pH. Such information will be used for generating the optimal combined solubility and permeability (i.e. Jss) in the aqueous system which will be used to provide an initial clue of desirable pH, solubility and permeability for the organic-aqueous solutions to be used as the desired suitable sublingual and IN sildenafil formulation and dose. In addition, sildenafil saturated solubility in various solvents was determined at about pH 4.0-6.0.

### Materials

Sildenafil citrate was purchased from Assian Chemical Industries Ltd (Israel) manufactured at Teva API (Israel).

Water used was obtained from Nanopure Water Filtration System (Barnstead Nanopure Diamond Life Sci UV/UF system (Cat #D119310 purchased form APS Water Servives Corporation (Lake Balboa, CA, USA).

Tadalafil (TAD) 5 mg tablets were purchased from Polpharma (Poland).

Acetonitrile ≥99.5% ACS (CAS No. 75-05-8) was purchased from VWR Chemicals BDH®.

Methanol (“MeOH”) was purchased from VWR Chemicals BDH®.

Ethanol 190-Proof (CAS No. 64-17-5) was purchased from EMD Millipore (Burlington, MA, USA).

Glycerin or glycerol (Lot 70K0044) was purchased from Sigma-Aldrich (St. Louis, MO, USA).

Calcium Lactate Pentahydrate (Lot SLCB7173) was purchased from Sigma-Aldrich (St. Louis, MO, USA).

Glacial Acetic Acid (Lot B21R026) was purchased from Alfa Aesar (Haverhill, MA, USA).

NMP (1-Methyl-2-Pyrrolidinone) (Lot 51K3683) was purchased from Sigma Aldrich (St. Louis, MO, USA).

Sodium Phosphate Monobasic Monohydrate (Phosphate Buffer) (Cat #BDH9298, Lot #19E0356407) was purchased from BDH Chemicals (Radnor, PA, USA).

NaOH (Sodium Hydroxide) (Cat #SB0617, Lot #C26S617ROS) was purchased from Biobasic Canada Inc. (Markham, Ontario, Canada)

Diethylene glycol monoethyl ether (Transcutol) (Cat #8.03127, Lot #S7591827 831)

MultiScreen-IP Filter Plate (Cat #MAIPN4550)

Sodium Phosphate Dibasic, Heptahydrate (Phosphate Buffer) (Cat #8210, Lot #BB-0680R)

Polyethylene glycol 400 (Cat #PX1286B-2, Lot #60297045) was purchased from EMD Millipore (Burlington, MA, USA).

HCl (Hydrochloric Acid) (Cat #320331; Lot #SHBG2435V)

Phosphate buffered saline (PBS, tablet) (Cat #P4417; Lot #SLCD5938) was purchased from Sigma-Aldrich (St. Louis, MO, USA).

o-Phosphoric Acid, 85% (HPLC) (Cat #A260)

Triethylamine (Cat #04884; Lot #011464) was purchased from Thermo Fisher Scientific (Waltham, MA, USA)

Syringe Filter w/0.2 um pore size Cellulose Acetate Membrane (Cat #28145-475) was purchased from VWR (Radnor, PA, USA).

Tween 20 and Tween 80 from EMD Millipore (Burlington, MA, USA)

### Equipment

The equipment used in the following experiment was the same as that described in EXAMPLE A2.

### Procedure

Aqueous sildenafil and multiple organic-aqueous sildenafil solutions were screened for saturated solubility. To determine the aqueous saturated solubility of sildenafil at different pH, excess amount of sildenafil citrate API was used and the solution was prepared at room temperature by the “shake flask” method with pH adjusted (at the range of pH 3.5-7.5) with use of a pH meter. Afterwards the solution was filtered using the VWR 0.2 mcron filter. The filtrate was subsequently determined by HPLC for sildenafil concentration was carried out using the method similar to that for vardenafil HPLC assay described in EXAMPLE A2 and B1 with tadalafil used as the internal standard.

### Results

The validity of the sildenafil HPLC assay was assessed in reference to the validated vardenafil assay (EXAMPLE A2) with respect to quality control sample and standard curves. Similar results were obtained and the lower limit of quantitation was 0.2 ug/ml.

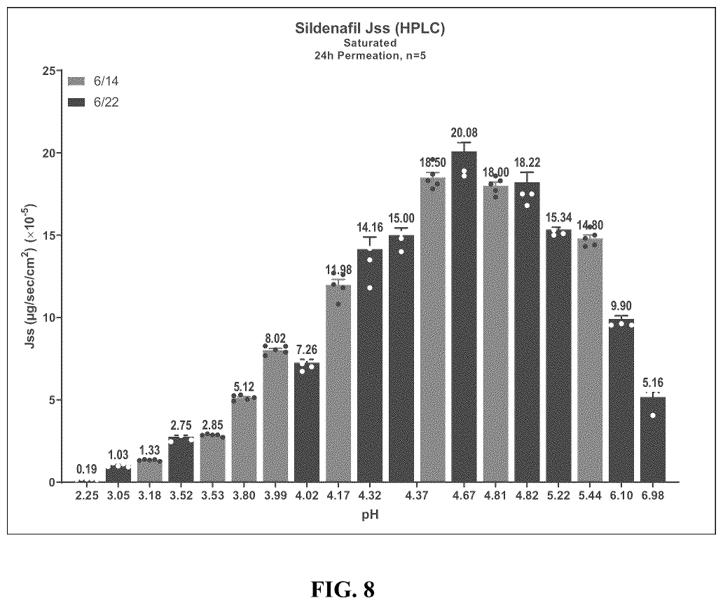

Table 10 shows relevant comparative saturated solubility of sildenafil in several solvents within the pH range of 3.5-7.5. Higher solubility occurs at lower pH and its solubility above pH 4.5 is greatly reduced. The comparison of sildenafil Jₛₛ in aqueous solution at different pH values is shown in FIG. 8. Based such data, JₛₛₚHₘₐₓ for sildenafil in water is estimated to be at pH4.6. However, the solubility representative organic aqueous solutions appear to be optimal at pH4.2-4.5 which correspond to within the range of sildenafil JₛₛₚHₘₐₓ (pH4.2-5.2, as shown in FIG. 8). For practical consideration of industrial application, Tables 11 and 12 show the saturated solubility of sildenafil at about pH 4.2 and pH4.5 in different solvents.

TABLE 10
Sildenafil saturated solubility in different solvents at different pH*

| | pH | | | | | |
|---|---|---|---|---|---|---|
| Sample Solutions | 3.5 | 4.0 | 4.5 | 5.0 | 6.0 | mg/ml |
| Water | 3.68 | 3.54 | 3.27 | 1.00 | 0.05 | mg/ml |
| Calcium Lactate(3.5%) | 6.93 | 8.62 | 7.13 | 0.44 | 0.1 | mg/ml |
| Acetic Acid (5%) | 10.69 | 12.86 | 14.83 | 1.05 | 0.27 | mg/ml |

*Determined at room temperature and atmospheric pressure

TABLE 11
Sildenafil saturated solubility in different solvents at about pH 4.2*

| | Saturated | | |
|---|---|---|---|
| Solution | Concentration | SD | N |
| Water | 3.54 | 0.63 | 28 |
| Calcium Lactate (3.5%) | 8.62 | 4.37 | 2 |
| Acetic Acid/Calcium Lactate (1%/3.5%) | 31.69 | 7.16 | 4 |
| Acetic Acid/Calcium Lactate (5%/3.5%) | 47.27 | | 1 |
| NMP (10%) | 6.98 | | 1 |
| Acetic Acid/NMP/Calcium Lactate (1%/10%/3.5%) | 49.20 | 2.25 | 4 |
| Acetic Acid/NMP/Calcium Lactate (5%/10%/3.5%) | 37.21 | 5.46 | 8 |
| Acetic Acid/NMP/Calcium Lactate (20%/20%/3.5%) | 90.89 | 12.66 | 2 |
| Acetic Acid/NMP/Calcium Lactate/Tween 20/EtOH | 55.77 | | 1 |
| (5%/10%/3.5%/5%/10%) | | | |
| Acetic Acid (1%) | 4.57 | | 1 |
| Acetic Acid (5%) | 12.86 | 0.91 | 2 |
| Acetic Acid/Calcium Lactate/Glycofurol (1%/3.5%/20%) | 51.22 | | 1 |
| Acetic Acid/NMP/Calcium Lactate/Tween 20 | 43.89 | | 1 |
| (5%/10%/3.5%/10%) | | | |
| Acetic Acid/NMP/Calcium Lactate/Tween 20 | 46.40 | | 1 |
| (5%/10%/3.5%/20%) | | | |
| Acetic Acid/NMP/Calcium Lactate/Tween 20/EtOH | 48.26 | | 1 |
| (5%/10%/3.5%/10%/10%) | | | |
| Acetic Acid/NMP/Calcium Lactate/Tween 20/EtOH | 44.83 | | 1 |
| (5%/10%/3.5%/20%/10%) | | | |
| Calcium Lactate/Tween 20/EtOH (3.5%/5%/10%) | 32.62 | | 1 |
| NMP/Calcium Lactate/Tween 20/EtOH | 37.42 | | 1 |
| (10%/3.5%/5%/10%) | | | |
| Propylene Glycol/Calcium Lactate/Tween 20/EtOH | 44.53 | | 1 |
| (10%/3.5%/5%/10%) | | | |
| Tween 20/EtOH (20%/10%) | 7.59 | | 1 |
| Tween 80/EtOH (20%/10%) | 6.33 | | 1 |

*Determined at room temperature and atmospheric pressure.

TABLE 12
Sildenafil saturated solubility in different solvents at about pH 4.4*

| | Saturated | | |
|---|---|---|---|
| Solution | Concentration | SD | N |
| Water | 3.27 | 1.07 | 28 |
| Acetic Acid (1%) | 3.22 | 0.65 | 3 |
| Calcium Lactate (3.5%) | 33.74 | 0.01 | 4 |
| NMP (10%) | 9.50 | 0.01 | 4 |
| NMP (25%) | 12.70 | | 1 |
| NMP (35%) | 12.50 | | 1 |
| Acetic Acid/NMP (5%/10%) | 29.03 | | 1 |
| Acetic Acid/NMP/Calcium Lactate | 61.11 | 0.01 | 5 |
| (5%/20%/3.5%) | | | |
| Acetic Acid/NMP/Calcium Lactate | 55.53 | 0.01 | 4 |
| (1%/10%/3.5%) | | | |
| Acetic Acid/NMP/Calcium Lactate | 26.81 | 0.25 | 2 |
| (5%/10%/3.5%) | | | |
| Acetic Acid/NMP/Calcium Lactate | 56.03 | | 5 |
| (5%/15%/3.5%) | | | |
| Acetic Acid/NMP/Calcium Lactate | 59.88 | 0.01 | 5 |
| (5%/25%/3.5%) | | | |
| Acetic Acid/Calcium Lactate (1%/3.5%) | 7.65 | 4.71 | 3 |
| Acetic Acid/Calcium Lactate (5%/3.5%) | 4.69 | 0.48 | 3 |
| NMP/Calcium Lactate (25%/5%) | 55.16 | 0.01 | 5 |
| NMP/Calcium Lactate (20%/5%) | 63.68 | 6.87 | 13 |
| NMP/Transcutol (40%/20%) | 27.23 | 0.15 | 3 |
| NMP/Transcutol/PEG400 (30%/20%/10%) | 23.04 | 3.41 | 3 |

*Determined at room temperature and atmospheric pressure

### Conclusion

As disclosed herein, certain organic-aqueous solvent mixtures, such as acetic acid/NMP/calcium lactate-aqueous mixture, can significantly enhance sildenafil solubility as compared to solubility in a pure aqueous solution (Tables 11 and 12). The solubility of sildenafil is pH-dependent. Based on our study results, saturated sildenafil solubility can be further enhanced by increasing the % organic solvent concentration.

If a minimum solubility of sildenafil 42 mg/ml desired for IN formulation(calculation shown in EXAMPLE D1), the sildenafil aqueous saturated solubility is below 5 mg/ml at pH4 which is not a suitable solvent for sildenafil IN formulation. However, a number of other organic-aqueous solvents or mixtures with solubility of at least 42 mg/ml at about pH4 have been identified (see Table 11 and 12) and are likely to be suitable for sildenafil IN formulation if their permeability values are similar to higher than that for sildenafil aqueous solutions (see Table 13 and 14 in EXAMPLE D3 below).

### Example D3

### Screening for Sildenafil Permeability and Flux Using PAMPA

This example describes the determination of permeability of sildenafil using a parallel artificial membrane permeability assay (PAMPA).

As disclosed herein, the permeability of sildenafil in different solvents at room temperature and atmospheric pressure was screened using in vitro PAMPA. The PAMPA predicts passive absorption of drugs (22-24). The unit of measurement is the apparent permeability (Papp) obtained at steady state, expressed as cm/s. Also another associated measurement is the flux (Jss) at a particular pH, expressed as the quantity of drug across the unit area per second, is calculated from the Papp and saturated solubility.

As disclosed herein, the effect of pH on the permeability of saturated aqueous sildenafil solution was determined. In addition, different organic-aqueous solution on improving permeability of saturated sildenafil was also determined at about pH4.2 and 4.6. Prior to our studies, the effect of pH on permeation of sildenafil in different organic-aqueous solutions was unknown and could not be accurately predicted.

### Materials and Equipment

The material and equipment are similar to that described in EXAMPLE B1 with sildenafil and solvents same as that described in D2.

### Procedure

### (a) Solution Preparation

Saturated solutions of sildenafil HCl trihydrate (3 ml) in different solvents were prepared by using an increasing amount of sildenafil and adjusted to the desired pH (range 3.5-6.0 with the use of a pH meter), as described in Example E2 above.

### (b) Permeation Studies Using PAMPA

Sildenafil permeation studies were performed using PAMPA with the receiver plate and multiscreen-IP filter plates as described for vardenafil under EXAMPLE B2. Sildenafil in different solvents at pH 4.2 and 4.5 are shown in Table 13 and 14.

### (c) HPLC Analysis

The concentrations of sildenafil in the donor or receiving chamber were prepared similar to that described for vardenafil and the HPLC assay was performed similar as that described in Example D2.

### Results

The mean Papp and Jss of sildenafil in different sildenafil solutions at pH4.2 and 4.5 are shown in Tables 13 and 14.

TABLE 13
Effect of solvents on sildenafil permeability and flux at about pH 4.2*

| | | Mean Papp | SD Papp | Mean Jss | SD Jss |
|---|---|---|---|---|---|
| Solution | N | (cm/s) | (cm/s) | (μg/sec/cm²) | (μg/sec/cm²) |
| Water | 28 | 3.27E−08 | 3.03E−08 | 1.08E−04 | 9.45E−05 |
| Calcium Lactate (5%) | 8 | 1.13E−07 | 1.22E−07 | 1.66E−03 | 1.78E−03 |
| NMP (25%) | 4 | 6.69E−09 | 4.40E−09 | 4.21E−05 | 2.77E−05 |
| Acetic Acid/Calcium Lactate | 4 | 2.13E−07 | 2.62E−07 | 5.99E−03# | 7.37E−03 |
| (1%/3.5%) | | | | | |
| Acetic Acid/Calcium Lactate | 1 | >2.1E−07 | n/a | >5.99E−03# | n/a |
| (5%/3.5%) | | | | | |
| Acetic Acid/NMP/Calcium | 4 | 7.76E−08 | 5.18E−08 | 3.73E−03# | 2.49E−03 |
| Lactate (1%/10%/3.5%) | | | | | |
| Acetic Acid/NMP/Calcium | 8 | 1.82E−07 | 1.00E−07 | 6.32E−03# | 2.91E−03 |
| Lactate (5%/10%/3.5%) | | | | | |
| Acetic Acid (20%) | 34 | 1.18E−09 | 3.35E−09 | 6.76E−05 | 1.92E−04 |
| Acetic Acid/Calcium Lactate/ | 5 | 3.79E−06 | 5.27E−06 | 2.11E−01# | 2.93E−01 |
| Tween 20/EtOH/Glycofurol | | | | | |
| (1%/3.5%/5%/10%/20%) | | | | | |
| Acetic Acid/NMP (20%/10%) | 24 | 2.75E−09 | 6.41E−09 | 1.30E−04 | 3.02E−04 |
| Acetic Acid/NMP (20%/25%) | 31 | 3.51E−09 | 9.53E−09 | 1.42E−04 | 3.85E−04 |
| Acetic Acid/NMP/Calcium | 4 | 4.06E−06 | 4.70E−06 | 1.88E−01# | 2.17E−01 |
| Lactate/Tween 20 | | | | | |
| (1%/10%/3.5%/5%) | | | | | |
| Acetic Acid/NMP/Calcium | 5 | 3.55E−06 | 4.94E−06 | 1.87E−01# | 2.61E−01 |
| Lactate/Tween 20/EtOH | | | | | |
| (1%/10%/3.5%/5%/10%) | | | | | |
| Acetic Acid/NMP/Calcium | 4 | 4.25E−06 | 8.51E−06 | 2.31E−01# | 4.63E−01 |
| Lactate/Tween 20/EtOH | | | | | |
| (5%/10%/3.5%/5%/10%) | | | | | |
| Calcium Lactate (3%) | 5 | 1.71E−07 | 9.57E−08 | 2.38E−03 | 1.33E−03 |
| Calcium Lactate/NMP (5%/20%) | 9 | 7.30E−08 | 4.47E−08 | 2.08E−03# | 1.07E−03 |
| NMP (20%) | 6 | 3.62E−08 | 4.00E−08 | 5.15E−04 | 5.69E−04 |

*Determined at room temperature and atmospheric pressure.
#Higher than Jss in water, or Jss(ref) and solubility >42 mg/mL.

TABLE 14
Effect of solvents on sildenafil permeability and flux at about pH 4.5*

| | | Mean Papp | SD Papp | Mean Jss | SD Jss |
|---|---|---|---|---|---|
| Solution | | (cm/s) | (cm/s) | (μg/sec/cm²) | (μg/sec/cm²) |
| Water | 28 | 6.64E−08 | 5.50E−08 | 1.83E−04 | 1.32E−04 |
| Calcium Lactate (3.5%) | 4 | 7.08E−08 | 8.27E−08 | 2.39E−03 | 2.79E−03 |
| Acetic Acid/NMP/Calcium Lactate | 5 | 2.16E−07 | 1.88E−08 | 1.32E−02 | 1.15E−03 |
| (5%/20%/3.5%) | | | | | |
| NMP/Calcium Lactate (20%/5%) | 13 | 7.51E−09 | 4.58E−09 | 4.59E−04 | 2.83E−04 |
| Acetic Acid/Calcium Lactate | 7 | 4.87E−07 | 4.39E−07 | 5.20E−03 | 1.18E−03 |
| (1%/3.5%) | | | | | |
| NMP (10%) | 4 | 2.16E−08 | 3.71E−09 | 2.05E−04 | 3.52E−05 |
| Acetic Acid/Calcium Lactate | 7 | 2.70E−06 | 1.45E−06 | 1.51E−02 | 9.59E−04 |
| (5%/3.5%) | | | | | |
| Acetic Acid/NMP/Calcium Lactate | 4 | 1.01E−07 | 2.30E−08 | 5.61E−03# | 1.28E−03 |
| (1%/10%/3.5%) | | | | | |
| Acetic Acid/NMP/Calcium Lactate | 5 | 2.35E−07 | 1.67E−08 | 1.31E−02# | 9.35E−04 |
| (5%/15%/3.5%) | | | | | |
| Acetic Acid/NMP/Calcium Lactate | 5 | 2.03E−07 | 1.46E−08 | 1.21E−02# | 8.76E−04 |
| (5%/25%/3.5%) | | | | | |
| NMP/Calcium Lactate (25%/5%) | 5 | 1.44E−08 | 4.39E−09 | 7.94E−04 | 2.42E−04 |
| Acetic Acid/NMP/Calcium Lactate | 22 | 1.95E−07 | 9.89E−08 | 9.67E−03 | 4.99E−03 |
| (5%/10%/3.5%) | | | | | |

*Determined at room temperature and atmospheric pressure.
#Higher than Jss in water, or Jss(ref) and solubility >42 mg/mL.

### Conclusion

As disclosed herein, sildenafil solubility is pH dependent. Sildenafil aqueous solution increases permeation/permeability with increasing pH (corresponding to higher % of unionized species theoretically expected as pH increases from 2.25 to 7.0 (FIG. 8). Drug flux of aqueous sildenafil appears optimal at around pH 4.5 or around pH 4.6.

Based on nasal spray volume of 150 ul or 0.15 ml per nostril or 0.3 ml for both nostrils, the Solmin required for one IN sildenafil dose is estimated to be 42 mg/ml (12.5 mg/0.3 ml=42 mg) (See calculation in EXAMPLE D1)

Since Jss (ref)=JsspHmax (Solmin/Sil(ssol)pHmax) (see equation derivation in EXAMPLE B1), at pHmax 4.5-4.6, sildenafil aqueous solubility is 3.34 mg/ml (Table 10) and JsspHmax is 1.83E-04 ug/s/cm2,

$$Jss ( ref ) = ( 1.83 E - 04 ) ( 42 / 3.34 ) = 2.3 E - 03 ug / s / cm 2.$$

Similarly if estimated at pHmax 4.2, sildenafil aqueous solubility is 3.54 mg/ml (Table 9) and JsspHmax is 1.08E-04 ug/s/cm2,

$$Jss ( ref ) = ( 1.08 E - 04 ) ( 42 / 3.54 ) = 1.3 E - 03 ug / s / cm 2.$$

Per formulation and methods described in EXAMPLES A1, A2, B1, and B2 any sildenafil organic-aqueous mixture with solubility of >42 mg/ml with a corresponding Jss value >Jss (ref) 1.3E-03 to 2.3E-03 ug/s/cm2 should qualify for the IN sildenafil formulation.

A number of organic-solvents with a solubility of >42 mg/ml at about pH4.2, have been discovered to meet or exceed the sildenafil Jss (ref) of 1.28E-03 ug/see/cm2, and can be considered as suitable formulation for IN sildenafil (see Table 11). Similarly, a number of organic-solvents with a solubility of >42 mg/ml at about pH 4.5, have been also noted to meet or exceed the sildenafil Jss (ref) of 2.3E-03 ug/see/cm2, and can be considered as suitable formulation for IN sildenafil (see Table 14), if the suitable candidates are further confirmed with Calu-3 studies.

### Example D4

### Screening for Permeability of Sildenafil Using Calu-3 Cell Line

This example describes the determination of permeability of sildenafil using a Calu-3 cell line model.

As disclosed herein, the permeability of sildenafil in different solvents at 37C and atmospheric pressure was screened using the in vivo cell line model, Calu-3 (a non-small-cell lung cancer line).

The Papp of aqueous soluble drugs determined by the Calu-3 cell line model has been shown to be related to the IN absorption in animal studies when determined at pH 7.4 (25-26). As disclosed herein, the Calu-3 cell line model was utilized for confirmation of the consistency of sildenafil Papp values of different organic-aqueous solutions in comparison to that in water

### Materials

The materials used were similar as that for the vardenafil study as described in EXAMPLE B2 and sildenafil solution described in E2.

### Equipment

The equipment used were similar as that for the vardenafil study as described in EXAMPLE B2

### Procedure

### (a) Solution Preparation

Saturated solutions of sildenafil (1.5 mg/mL) in 6 different selected solvents were prepared and adjusted to about pH4.2 or pH 4.5 as described previously. After the sildenafil powder is dissolved the solution is further mixed overnight on a rotating platform. Afterwards, the solutions were filtered using a 0.2 m filter. The filtrates were then used for the permeation studies.

### (b) Culture of Calu-3 and Preparation of Monolayers

The culture was carried out similar to that described previously [25-26] and in EXAMPLE B2.

### (c) In-Vitro Permeation/Permeability Study Using Calu-3 Cell Line Model

The TEER determination, growth medium and procedure were similar as that described in EXAMPLE B2.

### (d) HPLC Assay Preparation and Measurement

50 μl samples from the receiving and apical chambers were either mixed with 50 μl of 50% MeOH and 10 ul internal standard or diluted with 50 μl medium and internal standard. Supernatant was taken after centrifugation for HPLC analysis. HPLC analysis was performed as outlined in D2.

### (e) Calculation of Papp

Papp, expressed in cm/sec, was calculated from the samples analyzed by HPLC using the equations described in EXAMPLE B2.

### Results

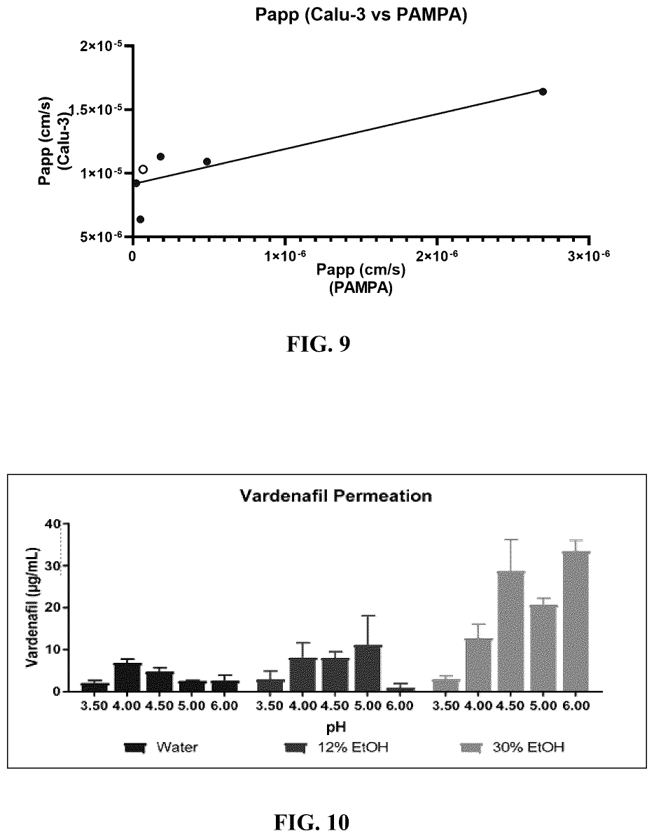

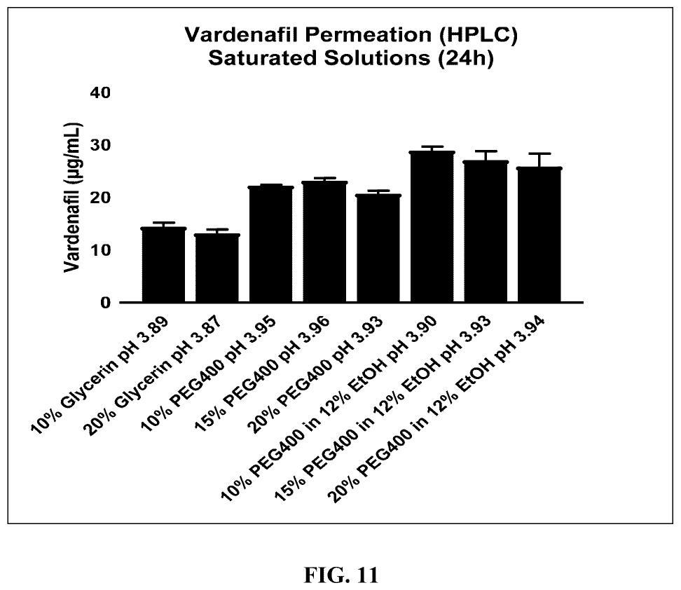

Comparison of sildenafil Papp by PAMPA to that of Calu-3 is shown in FIG. 9. In view of small number of data points considerable scattering of points are observed around the best-fit line. As the purpose of the plot is to compare Papp of sildenafil (obtained in organic-aqueous solution) to that obtained in aqueous solution by both PAMPA and Calu-3 methods, a value significantly lower than Papp from sildenafil aqueous solution can be easily identified at left lower quadrant. All other points represent Papp values which are either similar or better than that obtained from aqueous solution. Although FIG. 9 shows one low point at lower left quadrant and it corresponds to be calcium lactate(3.5%)-aqueous solution, the magnitude is not big and there is no statistical significantly difference comparing sildenafil Papp in aqueous solution vs that from calcium lactate(3.5%)-aqueous solution determined by PAMPA or Calu-3 respectively.

### Conclusion

The relative Papp values of various solutions also directly reflect relative Jss values since the concentration of each solution is 1.5 mg/ml.

Since sildenafil PAMPA Papp at pH 4.2 is close to that at pH 4.5, a similar relationship of PAMPA Papp at pH 4.2 vs Calu-3 Papp as that shown for pH 4.5 (FIG. 9) is expected. Based on sildenafil solubility and Papp requirements in organic-aqueous solutions as proposed by the current method and the solubility and permeability results of sildenafil in various organic-aqueous solutions, sildenafil in acetic acid/calcium lactate(1%/3.5%)-aqueous solution, acetic acid/calcium lactate(5%/3.5%)-aqueous solution, and acetic acid/NMP/calcium lactate(5%/10%/3.5%)-aqueous solution at pH 4.2 or pH 4.5 is expected to be a suitable sildenafil IN formulation,

### Example D5

### Comparison of IN Vs Oral Administration of Select Phosphodiesterase Inhibitors in Rats

This example describes (1) the improved sildenafil bioavailability following appropriate formulation administered by IN as compare to oral route, and (2) the confirmation of the current proposed method in identifying specific solubility, Papp, and concentration needed for IN sildenafil formulations,

IN administration allows compounds to bypass liver metabolism and together with rapid transmucosal permeation (if using appropriate formulation), can lead to quicker absorption (faster peak concentration time or Tmax), higher peak concentration (Cmax) and greater bioavailability compared to oral administration. Sildenafil has high first pass liver metabolism and can benefit from IN administration. Appropriate formulations will be essential to achieve the improved Cmax, Tmax and bioavailability from IN administration. Thus specific solubility, Papp, and concentration needed for IN sildenafil formulations as per current method will be needed. Prior to the study, the effect of certain organic-aqueous formulations (with aqueous content greater than 50%) on sildenafil solubility and Papp suitable for IN formulation is unknown. The present work under D5 is to confirm the application of the proposed method for sildenafil in achieving superior results from IN formulation.

### Procedure

Sildenafil aqueous solution and organic-aqueous solutions were prepared using the same protocol as described previously. Sprague Dawley rats with jugular vein cannula inserted were ordered from Envigo RMS, Inc. (Indianapolis, IN). After arrival, the rats were adjusted to the animal vivarium environment of the Western University of Health Sciences for one week before the pharmacokinetic study. The anesthesia and study procedure were carried out similar to a previously published study (29). A total of 3 sildenafil formulations were selected based on it solubility, permeability and concentration required for suitable IN formulation as per proposed method. All 3 formulations (see Table) were administered by IN route and one by oral (PO) route via gavage.

As per rat study described for vardenafil (see EXAMPLE C2), the administration of the formulations was randomized, with 2 rats received each treatment and then crossed over after one week. A 200 uL of blood was obtained from each rat at 0, 2, 5, 10, 15, 20, 30, 45, 60, 120, 180 minutes (the 2 minute sample was skipped in the rats receiving oral formulation. After completion of any blood sampling, the samples were centrifuged and plasma collected and stored for analysis using Liquid Chromatography Tandem Mass Spectrometry (LC/MS/MS, Sciex API4000 and Agilent HPLC 1200 system) The assay used rat plasma to construct the standard curve and Tadalafil was used as the internal standard (IS). The assay procedure was similar to vardenafil assay (see Determination of the plasma concentration of sildenafil under EXAMPLE C3)

### Results

The standard curve for the assay of the sildenafil concentration determination in rat plasma showed excellent correlation coefficient (R²=0.9924-0.9976 for the concentration of 2 ng/ml-500 ng/ml). The accuracy of the quality control (QC) samples ranged from 96-100% for the 4 QC samples tested. The precision or CV ranged from 1.9-4.6%. The sildenafil formulation and number of rats received the IN and PO dosages are shown in Table 15. The Pharmacokinetics of IN and orally administered formulation are shown in Table 16. As shown in the Table 16, the Cmax and AUC are significantly higher with the IN formulations administered by IN route compared to the one formulation administered by the oral route, when normalized to mg/kg dose. For the same formulation administered by IN vs oral routes, the IN administration resulted in 6-fold higher AUC. The Tmax was also significantly shorter by IN route compared to oral route. The mean differences in Cmax or Tmax between IN vs oral administration are more than 10 fold. No significant difference in these parameters was observed among the 3 formulations administered by IN route.

TABLE 15
Formulations containing sildenafil administered
to rats either PO or IN

| # | Formulation | N | Route | dose |
|---|---|---|---|---|
| F1 | acetic acid/calcium lactate (5%/3.5%) | 5 | IN | 0.84 mg |
| F1 | acetic acid/calcium lactate (5%/3.5%) | 4 | PO | 1.69 mg |
| F2 | acetic acid/NMP/calcium lactate | 6 | IN | 0.76 mg |
| | (5%/10%/3.5%); | | | |
| F3 | acetic acid/calcium lactate (1%/3.5) | 4 | IN | 0.76 mg |

TABLE 16
Pharmacokinetic parameters of sildenafil as nasal spray and oral tablet in rat (data expressed as mean ± SD)

| | | Oral | Nasal | Nasal | Nasal |
|---|---|---|---|---|---|
| Parameter | Unit | F1 | F1 | F2 | F3 |
| T_{1/2} | min | 64.0 ± 22.1 | 43.1 ± 10.1 | 49.7 ± 21.9 | 40.2 ± 10.1 |
| Tₘₐₓ | min | 65.0 ± 37.9 | 3.8 ± 1.6 | 4.5 ± 1.2 | 6.3 ± 2.5 |
| Cₘₐₓ | ng/mL | 73.2 ± 61.5 | 715.6 ± 245.2 | 713.6 ± 68.7 | 415.9 ± 148.9 |
| AUC₀₋ₜ | ng/ml · min | 6473.0 ± 6704.9 | 18589.8 ± 2954.9 | 22619.3 ± 6191.9 | 15458.5 ± 8153.8 |

Dose normalization to 1 mg/kg

| | | | | | |
|---|---|---|---|---|---|
| Cₘₐₓ | (ng/ml)/(mg/kg) | *14.6 ± 12.4 | 280.8 ± 94.9 | 312.5 ± 30.4 | 171.6 ± 57.3 |
| AUC₀₋ₜ | (ng/ml · min)/(mg/kg) | *1291.6 ± 1355.2 | 7783.2 ± 1086.3 | 9982.7 ± 2733.2 | 6360.2 ± 3213.2 |

F1 = acetic acid/calcium lactate (5%/3.5%);
F2 = acetic acid/NMP/calcium lactate (5%/10%/3.5%);
F3 = acetic acid/calcium lactate (1%/3.5%);
*P < 0.05 when comparing IN treatment with oral treatment

### Discussion and Conclusion

The results of the relative bioavailability (AUC), Cmax (peak concentration) and Tmax (time of Cmax) between IN and oral route are significantly different and are consistent with that expected from IN formulations identified by present new method described in EXAMPLES A1, A2, B1, B2. Thus, these rat data further confirm the validity and utility of the present method of identifying desired IN formulations.

### Example E

### Formulation to Enhance Permeation and Flux of One or More Phosphodiesterase Inhibitor and Other Ionizable Basic/Acidic Drugs Across a Mucosal Membrane

This example describes the formulation for enhancing solubility and permeability (flux) of one or more phosphodiesterase inhibitor across a mucosal membrane.

As disclosed herein, based on methods described in EXAMPLES A1,A2,B1,B2, the formulation identified comprises the phosphodiesterase inhibitor vardenafil dissolved in an organic-aqueous solvent comprising ethanol and PEG400 at pH 4.0, wherein the organic-aqueous solvent enhances solubility of the phosphodiesterase inhibitor relative to solubility of the phosphodiesterase inhibitor in water. This formulation comprises ethanol at 12% as a component. In other alternatives, the formulation may comprise any alcohol, such as glycerol, and may be present at any concentration from 5% to 40%, including 25% and 30%. As disclosed herein, the organic-aqueous solvent of this formulation comprises another component, PEG400 at 15%. In other alternatives, the formulation may comprise any polyether or polyethylene glycol, such as PEG 6000, at a concentration between 1% to 20%. As disclosed herein, the formulation is at pH 4.0. In other alternatives, the formulation may be at any pH from 3.5 to 7.5. As disclosed herein, the phosphodiesterase inhibitor of the formulation is vardenafil. In other alternatives, the formulation may comprise other drugs in suitable organic-aqueous solutions identified per formulation and methods described similar to examples A1, A2, B1, and B2. The formulation will then be administered intranasally to a subject for treating erectile dysfunction or other diseases as appropriate. The intranasal administration will allow the formulation to contact the subject's mucosal membrane. In other alternatives, the mucosal membrane is contacted with the formulation through sublingual administration to the subject.

With respect to the use of substantially any plural and/or singular terms herein, those having skill in the art can translate from the plural to the singular and/or from the singular to plural as is appropriate to the context and/or application. The various singular/plural permutations can be expressly set forth herein for sake of clarity.

Unless the context requires otherwise, throughout the present specification and claims, the word “comprise” and variations thereof, such as, “comprises” and “comprising,” which is used interchangeably with “including,” “containing,” or “characterized by,” is inclusive or open-ended language and does not exclude additional, unrecited elements or method steps.

The phrase “consisting of” excludes any element, step, or ingredient not specified in the claim.

The phrase “consisting essentially of” limits the scope of a claim to the specified materials or steps and those that do not materially affect the basic and novel characteristics of the claimed invention. The present disclosure contemplates embodiments of the invention formulations, compositions, and methods corresponding to the scope of each of these phrases. Thus, a formulation, composition, or method comprising recited elements or steps contemplates particular embodiments in which the formulation, composition, or method consists essentially of or consists of those elements or steps.

Wherever a method of using a composition or formulation (e.g., a method of treating erectile dysfunction, comprising administering a formulation or comprising contacting a mucosal membrane with a formulation) is disclosed herein, the corresponding composition or formulation for use is also expressly contemplated. For example, for the disclosure of a method of treating erectile dysfunction, comprising administering a formulation or comprising contacting a mucosal membrane with a formulation comprising one or more phosphodiesterase inhibitor in an organic-aqueous solvent, the corresponding composition or formulation for treating erectile dysfunction is also contemplated.

Reference throughout this specification to “one embodiment” or “an embodiment” or “an aspect” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment.

Furthermore, the particular features, structures, or characteristics may be combined in any suitable manner in one or more embodiments.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A formulation for enhancing permeation of vardenafil across a nasal mucosal membrane, comprising:
(a) vardenafil; and
(b) an organic-aqueous solvent comprising an alcohol, a polyether, diethylene glycol monoethyl ether, a medium chain glyceride, one or more saturated polyglycolyzed C8-C10 glyceride, or a combination thereof;
(c) wherein the formulation has a pH of about 3.5 to about 4.5 and wherein the organic-aqueous solvent enhances solubility of the vardenafil relative to solubility of the vardenafil in water.

2. The formulation of claim 1, wherein the organic-aqueous solvent comprises an alcohol.

3. The formulation of claim 2, wherein the alcohol is ethanol or glycerol.

4. The formulation of claim 3, wherein the ethanol is present at a concentration of 5% to 40%.

5. (canceled)

6. The formulation of claim 1, wherein the organic-aqueous solvent comprises a polyether.

7. The formulation of claim 6, wherein the polyether is polyethylene glycol.

8. The formulation of claim 7, wherein the polyethylene glycol is PEG 6000 or PEG 400.

9. The formulation of claim 7, wherein the polyethylene glycol is present at a concentration of 1% to 20%.

10. (canceled)

11. (canceled)

12. The formulation of claim 1, wherein the vardenafil is provided in combination with one or more other active ingredients for treating erectile dysfunction.

13. The formulation of claim 12, wherein the one or more other active ingredients comprise another phosphodiesterase inhibitor.

14. The formulation of claim 13, wherein the other phosphodiesterase inhibitor is selected from the group consisting of sildenafil and tadalafil.

15. (canceled)

16. A method of treating erectile dysfunction of a subject in need thereof, comprising contacting a nasal mucosal membrane of the subject with a formulation of claim 1, thereby treating the erectile dysfunction of the subject.

17. The method of claim 16, wherein contacting the mucosal membrane comprises intranasal administration.

18. (canceled)

19. A method of preparing a formulation according to claim 1, comprising:
adding the vardenafil to the organic-aqueous solvent; and
adjusting the pH of the organic-aqueous solvent comprising the vardenafil to about 3.5 to about 4.5.

20. The method of claim 19, wherein solubility of the vardenafil is increased in the organic-aqueous solvent relative to solubility of the vardenafil in water.

21. The method of claim 19, wherein permeation of the vardenafil across a nasal mucosal membrane is increased in the organic-aqueous solvent relative to permeation of the vardenafil in water.

22. The method of claim 19, wherein bioavailability of the vardenafil is increased in the organic-aqueous solvent relative to bioavailability of the vardenafil in water.

23. The method of claim 19, wherein the organic-aqueous solvent comprises an alcohol.

24. The method of claim 23, wherein the alcohol is ethanol or glycerol.

25. The method of claim 24, wherein the ethanol is present at a concentration of 5% to 40%.

26. (canceled)

27. The method of claim 19, wherein the organic-aqueous solvent comprises a polyether.

28. The method of claim 27, wherein the polyether is polyethylene glycol.

29. The method of claim 28, wherein the polyethylene glycol is PEG 6000 or PEG 400.

30. The method of claim 28, wherein the polyethylene glycol is present at a concentration of 1% to 20%.

31. (canceled)

32. (canceled)

33. The method of claim 19, wherein the vardenafil is combined with another active agent for treating erectile dysfunction.

34. The method of claim 33, wherein the other active agent is another phosphodiesterase inhibitor.

35. The method of claim 34, wherein the other phosphodiesterase inhibitor is selected from the group consisting of sildenafil and tadalafil.

36. (canceled)

37. (canceled)

38. (canceled)
